(19) United States
(12) Patent Application Publication
(10) Pub. No.: US 2025/0297215 A1
(43) Pub. Date: Sep. 25, 2025

(54) METHOD FOR PRODUCING MILK LIKE PRODUCTS

(71) Applicant: SOCIETE DES PRODUITS NESTLE S.A., Vevey (CH)

(72) Inventors: MARIA MARQUES DE LIMA, Prilly (CH); ARIANNA BIANCHI, Lonay (CH); OMID MASHINCHIAN, Lausanne (CH); MARINE KRAUS, Vuarrens (CH); FREDERIC DESTAILLATS, Servion (CH)

(21) Appl. No.: 18/705,367

(22) PCT Filed: Oct. 27, 2022
(86) PCT No.: PCT/EP2022/080089

§ 371 Date: Apr. 26, 2024

(30) Foreign Application Priority Data
Oct. 27, 2021 (EP) 21205070.2

(51) Int. Cl. C12N 5/071; C12N 5/00; C12N 5/0775; C12P 1/00

(52) U.S. Cl. CPC C12N 5/0631; C12N 5/0062; C12N 5/0668; C12P 1/00; C12N 2501/119; C12N 2501/12; C12N 2501/155; C12N 2501/33; C12N 2501/385; C12N 2501/91; C12N 2506/1392; C12N 2513/00

(57) ABSTRACT

A method for producing mammary gland cells and a method of producing a mammalian milk like product, for example a human milk like product, comprising generating lactocytes derived from mammalian adult breast milk stem cells (mBSC), for example from human adult breast milk stem cells (hBSC) and expressing the mammalian milk like product, for example the human milk like product from lactocytes.

### CROSS REFERENCE TO RELATED APPLICATIONS

The present application is a National Stage of International Application No. PCT/EP2022/080089, filed on Oct. 27, 2022, which claims priority to European Patent Application No. 21205070.2, filed on Oct. 27, 2021, the entire contents of which are being incorporated herein by reference.

### SEQUENCE LISTING

The instant application contains a Sequence Listing which has been submitted electronically in XML file format and is hereby incorporated by reference in its entirety. Said XML copy, created on Nov. 7, 2024, is named 3712036-01125_SL.xml and is 2,773 bytes in size.

### FIELD OF THE INVENTION

The present invention concerns a method for producing mammary gland cells. The present invention also concerns a method for producing in vitro a mammalian milk like product, for example a human milk like product, which comprises generating lactocytes derived from mammalian adult breast milk stem cells (hBSC), for example human adult breast milk stem cells (hBSC) through culture and differentiation and/or mammary-like gland organoids comprising such lactocytes and expressing the mammalian milk like product, for example the human milk like product, from such lactocytes and/or mammary-like gland organoids. The present invention also relate to the mammalian milk like product, for example human milk like product, obtainable from such method.

### BACKGROUND OF THE INVENTION

Mammalian and especially human milk is a complex fluid with a multitude of components, each of which may contribute substantially to infant and perhaps maternal health. It is becoming increasingly clear that human breastmilk is the most appropriate source of nutrition at least up to the age of 6 months. Many components of human milk are simply not found or poorly found or less active in cow's milk upon which infant formula manufacture is based. This includes for instance protein lactoferrin, growth factors, long chain polyunsaturated fatty acids or oligosaccharides. Human milk composition has been used as a gold standard to develop current infant formula, despite recent major development in infant formula composition, it is illusory to think that human milk replication will be achieved with current manufacturing processes.

Today the only source of human milk is human donors (breastfeeding mothers). Donation are reported for non-commercial use (human milk biobank) and commercial use. However, this is limited and has strong regulatory, safety and sometime ethical or religious constraints.

Stem cells were found in mammalian and especially human milk called human adult breastmilk stem cells (hBSC). hBSCs were shown to be highly plastic and to differentiate in culture into multiple cell types and more importantly into the three lineages required to shape the lobulo-alveolar structure of the human mammary gland (Hassiotou F. et al. Stem Cells. 2012). However, such document doesn't demonstrate the functionality of such cells.

Accordingly, it is an object of the present invention to provide improved methods for producing mammary gland cells and to reproduce expression of mammalian milk, for example human milk in cultured cells. It is also an object of the present invention to prepare customized mammalian milk like product, for example human milk like product from cultured cell secretions which could be adapted to specific needs of the recipient and/or to produce human milk bioactives to complement existing cow-based solutions for infant nutrition.

### SUMMARY OF THE INVENTION

The present invention solves the above mentioned technical problem.

Provided herein is a method of producing a population of mammary gland cells, comprising:
i) culturing mammalian breast milk stem cells (mBSCs) in a culture medium comprising bone morphogenic protein 4 (BMP4) and/or retinoic acid (RA) to generate embryoid bodies (EBs), and
ii) growing the EBs to generate a population of mammary cells.

Also provided herein is use of BMP4 and/or RA for increasing the differentiation efficiency of mammalian breast milk stem cell (mBSCs) into mammary-gland progenitor cells in a differentiation protocol.

Also provided herein is a method for producing a mammalian milk like product, comprising:
A) Generating lactocyte mammary-like gland organoids derived from mammalian breast milk stem cells (mBSC);
B) Secreting the mammalian milk like product from said lactocytes,
wherein step A) comprises culturing the hBSCs in a culture medium comprising BMP4 and/or RA.

Also provided herein is a human milk like product which is obtainable according to the methods described anywhere herein.

Also provided herein is a human milk like product according to the methods described anywhere herein for use in therapy.

Also provided herein is use of a human milk like product according to the methods described anywhere herein as a human milk substitute, optionally a breast-feeding substitute.

### DETAILED DESCRIPTION OF THE INVENTION

### Definitions

Within the context of the present invention, the term “in vitro” means performed or taking place in a test tube, culture dish, bioreactor or elsewhere outside a living organism.

Within the context of the present invention, the term “mammalian” identify an animal belonging to the mammalian species, for example human, cow, monkey, camel, sheep, goat etc.

Within the context of the present inventions, the term “lactocytes” or “mammary-like cells” identifies secretory epithelial cells expressing CK18 cell marker and derived from mammalian breast milk stem cells (mBSC) and in particular human adult breast milk stem cells (hBSCs). Human adult breast milk stem cells may be obtained from donors under appropriate informed consent. In one embodiment of the present invention, the BSC are not engineered. In one embodiment, they are not engineered to comprise an exogenous nucleic acid and/or an inducible gene expression system which includes an exogenous nucleic acid, where the inducible gene expression system is configured to express a hormone or a signaling factor. In one embodiment, the exogenous nucleic acid and/or inducible gene expression system which includes an exogenous nucleic acid is promoting the cell differentiation towards lactocytes.

Within the context of the present invention the term “mammary gland like organoids” or “mammary like organoids” means a miniaturized and simplified version of a mammary gland which develops in two or three dimensions (2D/3D) and which comprises lactocytes as above defined.

Within the context of the present invention, the term “human milk like product” is a cell cultured milk product. It is an edible product which is expressed by the lactocytes and/or mammary gland like organoids generated according to the process of the present invention.

The “human milk like product” according to the present invention can have the same components as human breast milk of a well-nourished mother (for example in terms of bioactives, macro and micronutrients and levels thereof). This is referred to herein as a “standard human milk product”. Alternatively, “human milk like product” according to the present invention can have altered ratios and concentrations of components found naturally in human breast milk of a well-nourished mother. This is referred to herein as a “non-standard milk like product”. A “human milk like product” according to the present invention can be modified such that it includes components that are not found naturally in human breast milk of a well-nourished mother (a “modified milk like product”). Non-limiting examples of human milk like products are selected from the group consisting of: supplement, fortifier, human breast milk substitute (or replacer) and ingredient enriched in only one and/or a portion of bioactives, macro- and micronutrients which can be typically found in human breast milk of a well-nourished mother.

The “human milk like product” can be used to replace consumption of naturally lactated milk (a “human milk substitute”). The milk substitute product can be used as a supplement (a “human milk supplement”) or as a fortifier (a “human milk fortifier”) to be consumed in combination with naturally lactated milk.

In an embodiment, the standard human milk like product according to the present invention comprises at least macro- and micronutrients which can be typically found in human breast milk of a well-nourished mother. In one embodiment, the standard human milk like product according to the present invention comprises: proteins, peptides, lipids (including linoleic acid and alpha-linolenic acid), carbohydrates, Vitamins (including Vitamin A, Vitamin D3, Vitamin E, Vitamin K, Thiamin, Riboflavin, Niacin, Vitamin B6, Vitamin B12, Pantothenic acid, folic acid, Vitamin C and Biotin), minerals (including iron, calcium, phosphorus, magnesium, sodium, chloride, potassium, manganese, iodine, selenium, copper and zinc), choline, myoinositol and L-carnitine. In one embodiment, the standard human milk like product according to the present invention also comprises at least one bioactive selected from the group consisting of: growth factors, cytokines, probiotics, extracellular vesicles (e.g. milk fat globules and or exosomes), bioactives from exosome (for example miRNA) and secretory IgA. The standard human milk like product according to the present invention is not the product of human breast lactation as occurring in nature.

In another embodiment, the human milk like product according to the present invention can be adapted to specific needs of the infant who will receive it. It may comprise only one and/or a portion of bioactives, macro and micro nutrients which can be typically found in human breast milk of a well-nourished mother. In such embodiment, the human breast milk like product may also be referred to with the term “non-standard human milk like product”. In one embodiment, the non-standard human milk like product according to the present invention comprises one or more of the nutrients or bioactives selected from the group consisting of proteins, peptides, lipids (including linoleic acid and alpha-linolenic acid), carbohydrates (including human milk oligosaccharides), Vitamins (including Vitamin A, Vitamin D3, Vitamin E, Vitamin K, Thiamin, Riboflavin, Niacin, Vitamin B6, Vitamin B12, Pantothenic acid, folic acid, myoinositol, L-carnitine, growth factors, cytokines, probiotics, extracellular vesicles (e.g. milk fat globules and or exosomes), bioactives from exosome (for example miRNA) and secretory IgA.

Within the context of the present invention, the term “non-modified human milk like product” indicates a human milk like product which is expressed by lactocytes and/or by the mammary gland like organoids generated according to steps A) and B) of the process of the present invention and which is not subject to the further treatment according to optional step C) of the process of present invention. Non-modified human milk like product may comprise both standard and non-standard human milk like products. Non limiting examples of non-standard human milk like products are selected from the group consisting of: supplement, fortifier, and ingredient enriched in only one and/or a portion of bioactives, macro and micro nutrients which can be typically found in human breast milk of a well-nourished mother.

Within the context of the present invention, the term “modified human milk like product” indicates a human milk like product which is expressed by lactocytes and/or by the mammary gland like organoids generated according to steps A) and B) of the process of the present invention and which is subject to the further treatment according to optional step C) of the process of present invention.

Modified human milk like products may comprise both standard and non-standard human milk like products.

Within the context of the present invention the term “EBs” means “embryoid bodies”.

Within the context of the present invention the term “mEBs” means “MammoCult medium-cultured embryoid bodies”.

MammoCult medium refers to a serum-free culture medium comprising basal medium, at least one proliferation supplement, heparin and hydrocortisone.

Within the context of the present invention the terms “embryoid bodies (EBs)”, “MammoCult medium-cultured embryoid bodies (mEBs)”, “mammospheres” and/or “spheroids” refer to three-dimensional aggregates formed in suspension by breast milk stem cells (BSC) under step A) of the process of the present invention.

The term “infant” in the context of the present invention identifies a child under the age of 12 months, such as under the age of 9 months, particularly under the age of 6 months.

In the context of the present invention the infant may be any term infant or preterm infant. In an embodiment of the invention, the infant is selected from the group of preterm infants and term infants.

The term “term infant” refers to infants born at term or at a gestational age of 37 weeks or more.

The term “preterm infant” refers to infants who are born at a gestational age of less than 37 weeks.

In the context of the present invention, the term “birth weight” means the first weight of the fetus or newborn obtained after birth.

Within the context of the present invention, the term “low birth weight” means a birth weight of less than 2500 g (up to and including 2499 g).

Within the context of the present invention, the term “very low birth weight” means a birth weight of less than 1500 g (up to and including 1499 g).

Within the context of the present invention, the term “extremely low birth weight” means a birth weight of less than 1000 g (up to and including 999 g).

The term “small for gestational age infant” refers to infants having a birth weight that is more than 2 standard deviations below the mean reference to a birth weight for gestational growth chart or having a birth weight that is less than the 10th percentile of population-based weight data obtained from infants at the same gestational age. The term “small for gestational age infants” includes infants who are small at birth either from a constitutive or genetic origin or, as a consequence of intrauterine growth restriction.

Within the context of the present invention, The term “young children” or “toddler” indicates a child between the age of 1 and 3 years.

The term “infant formula” as used herein refers to a nutritional composition intended for infants and as defined in Codex Alimentarius, (Codex STAN 72-1981) and Infant Specialities (incl. Food for Special Medical Purpose) as defined in Codex Alimentarius, (Codex STAN 72-1981). It also refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). The infant formulas encompass the starter infant formulas and the follow-up or follow-on formulas. Generally, a starter formula is for infants from birth as breast-milk substitute, and a follow-up or follow-on formula from the 6th month onwards.

The “growing-up milks” (or GUMs) are given from one year onwards. It is generally a milk-based beverage adapted for the specific nutritional needs of young children. They are nutritional compositions used for feeding children from 12 months to 2-3 years old in combination with other foods.

Within the context of the present invention, the term “fortifier”” refers to a composition which comprises one or more nutrients having a nutritional benefit for infants or young children.

By the term “milk fortifier”, it is meant any composition used to fortify or supplement either human breast milk, infant formula, growing-up milk or human breast milk fortified with other nutrients. Accordingly, the human milk fortifier of the present invention can be administered after dissolution in human breast milk, infant formula, growing-up milk or human breast milk fortified with other nutrients or otherwise it can be administered as a stand alone composition.

When administered as a stand-alone composition, the human milk fortifier of the present invention can be also identified as being a “supplement”. In one embodiment, the milk fortifier of the present invention is a supplement.

By the term “human milk fortifier”, it is meant any composition used to fortify or supplement human breast milk, or human breast milk fortified with other nutrients. The “human milk fortifier” according to the present invention may be intended to be administered to infants who were born preterm, with very low birth weight (VLBW) or with extremely low birth weight (ELBW).

The milk fortifier according to the present invention may be in powder of liquid form.

Milk fortifier compositions having a liquid form presents some particular benefits. For example, liquid formulations might be more convenient if coupled with a packaging that delivers calibrated drops of a certain weight or volume.

In addition, liquid formulations are easier to mix with the compositions to be fortified, whereas the powder ones can, in some cases, form lumps.

Within the context of the present invention, the term ‘increasing differentiation efficiency and maturation of human adult breast milk stem cells (hBSCs) into mammary-gland progenitor cells’ means increasing the proportion of mammary-gland progenitor cells compared to non-mammary-gland progenitor cells, generated from a starting population of hBSCs which have undergone a differentiation protocol. In the present invention, said differentiation protocol includes the use of BMP4 and/or RA. Thus, said increase of the proportion of mammary-gland progenitor cells may be compared to the proportion of mammary-gland progenitor cells relative to non-mammary-gland progenitor cells, generated from a starting population of hBSCs which have undergone a differentiation protocol that does not include the use of BMP4 and/or RA but is otherwise the same.

Within the context of the present invention, the term ‘increasing viability’ means increasing the number of cells that are live and healthy. Said cells are capable of further differentiation steps, for example in the context of the present invention, are capable of developing into mammary organoids.

Within the context of the present invention, the term ‘mammary-gland progenitor cells’ or similar means cells that express at least two mammary-gland progenitor markers. Said markers include but are not limited to CD49f, EpCAM, MUC1 and GATA3. Conversely, within the context of the present invention, the term ‘non mammary-gland progenitor cells’ or similar means cells that do not express at least two mammary-gland progenitor markers.

Reference herein to EpiCult or EpiCultB medium refers to a serum free culture medium comprising hydrocortisone, insulin, FGF10 and HGF.

A culture medium as disclosed anywhere herein refers to a solid, semi-solid or liquid comprising essential nutrients, designed to support the growth and differentiation of microorganisms. MammoCult medium is one example of a culture medium that may be used in the present invention.

### Method and Uses According to the Present Invention

The present invention relates to methods of producing mammary gland cells using hBSCs that are cultured and differentiated in specific conditions and using said mammary gland cells in methods for producing a mammalian milk like product in vitro.

It has been surprisingly demonstrated in the present invention that the addition of Bone morphogenetic protein 4 (BMP4) and/or Retinoic Acid (RA) in the methods as described anywhere herein, provides a more homogenous mixture of non-neural ectoderm progenitor cells by increasing the proportion of non-neural ectoderm progenitor cells and decreasing the proportion of neural ectoderm progenitor cells.

It has also been demonstrated in the present invention that the addition of BMP4 and/or RA in the methods as described anywhere herein increases the differentiation efficiency of hBSCs to mammary gland progenitor cells, and thus increasing the number of mammary gland progenitor cells produced by the methods as described anywhere herein. The benefits of this include a higher yield of mammary gland cells.

It has also been surprisingly found that the addition of RA in the methods as described anywhere herein, increases the viability of mammary gland progenitor cells produced by the methods of the invention.

Typically, serum-supplemented cultures are used to aid hBSC differentiation and morphogenesis. It has now been found that RA may be used as an effective alternative to serum. Thus, in some embodiments, the methods as described anywhere herein are advantageously serum-free.

It has been also demonstrated that BMP4 and RA in combination improve expression and secretion levels of milk-specific bioactive markers such as osteopontin (OPN).

It has also been demonstrated that because of the increased efficiency of differentiation due to the combination of BMP4 and RA, the overall differentiation protocol can be shortened in length. This has significant benefits including reduce cell death, higher milk like yields, and cost savings.

### Mammalian Mammary Cell Production

In one aspect, the present invention relates to methods of producing mammary gland cells using hBSCs that are cultured and differentiated in specific conditions.

Thus, the invention provides a method of producing a population of mammary gland cells, comprising:
i) culturing human adult breast milk stem cells (hBSCs) in a culture medium comprising bone morphogenic protein 4 (BMP4) and/or Retinoic Acid (RA) to generate embryoid bodies (EBs), and
ii) growing the EBs to generate a population of mammary cells.

The invention also provides use of BMP4 and/or RA for increasing the differentiation efficiency of human adult breast milk stem cells (hBSCs) into mammary-gland progenitor cells in a differentiation protocol.

Thus, the invention provides a method of producing a population of mammary gland cells, comprising:
i) culturing human adult breast milk stem cells (hBSCs) in a culture medium comprising bone morphogenic protein 4 (BMP4) to generate embryoid bodies (EBs), and
ii) growing the EBs to generate a population of mammary cells.

The invention also provides use of BMP4 for increasing the differentiation efficiency of human adult breast milk stem cells (hBSCs) into mammary-gland progenitor cells in a differentiation protocol.

Thus, the invention provides a method of producing a population of mammary gland cells, comprising:
i) culturing human adult breast milk stem cells (hBSCs) in a culture medium comprising Retinoic Acid (RA) to generate embryoid bodies (EBs), and
ii) growing the EBs to generate a population of mammary cells.

The invention also provides use of RA for increasing the differentiation efficiency of human adult breast milk stem cells (hBSCs) into mammary-gland progenitor cells in a differentiation protocol.

Thus, the invention provides a method of producing a population of mammary gland cells, comprising:
i) culturing human adult breast milk stem cells (hBSCs) in a culture medium comprising bone morphogenic protein 4 (BMP4) and Retinoic Acid (RA) to generate embryoid bodies (EBs), and
ii) growing the EBs to generate a population of mammary cells.

The invention also provides use of BMP4 and RA for increasing the differentiation efficiency of human adult breast milk stem cells (hBSCs) into mammary-gland progenitor cells in a differentiation protocol. In some embodiments, the invention also provides the use of BMP4 and RA for increasing the efficiency of producing a mammalian milk like product.

In some embodiments, the mammary gland cells form lactocyte mammary-like gland organoids. Said lactocyte mammary-like gland organoids are lactogenic, i.e., are capable of producing milk.

### Bone Morphogenic Protein 4 (BMP4)

In some embodiments, BMP4 is added to the culture medium in the early stages of the differentiation methods as described anywhere herein. In some embodiments, BMP4 is added to the culture medium between day 0 and day 10. In some embodiments, BMP4 is added to the culture medium between day 0 and day 6. In some embodiments, BMP4 is added to the culture medium between day 0 and day 3. Day 0 is the time point where the hBSCs are first added to the culture medium, i.e. when the hBSCs are first induced for differentiation.

In some embodiments, BMP4 is added to the culture medium for 3 days.

In some embodiments, BMP4 is added to the culture medium in a concentration of 5 to 20 ng/ml. In some embodiments, BMP4 is added to the culture medium in a concentration of 5 ng/ml. In some embodiments, BMP4 is added to the culture medium in a concentration of 10 ng/ml. In some embodiments, BMP4 is added to the culture medium in a concentration of 20 ng/ml.

In some embodiments, BMP4 is added to the culture medium between day 0 and day 3, and in a concentration of between 5 to 20 ng/ml.

The EBs generated in the methods as described anywhere herein express one or more mammary gland positive progenitor-cell markers. In some embodiments, said one or more mammary gland positive progenitor-cell markers are selected from EpCAM, CD49f, MUC1 and GATA3.

In some embodiments, the EBs have increased expression of one or more mammary gland positive progenitor-cell markers compared to the expression level of said mammary gland positive progenitor-cell markers in EBs not treated with BMP4.

In some embodiments, at least 10%, 20%, 30%, 40%, 50%, 60%, 70%, 80% or more of the EBs express one or more mammary gland positive progenitor-cell markers. In some embodiments, at least 10%, 20%, 30%, 40%, 50%, 60%, 70%, 80% or more of the EBs express two or more mammary gland positive progenitor-cell markers. In some embodiments, said mammary gland positive progenitor-cell markers are selected from EpCAM, CD49f, MUC1 and GATA3.

In some embodiments, at least 50% of EBs express EpCAM and CD49f mammary gland positive progenitor-cell markers. In some embodiments, BMP4 is added to the culture medium in a concentration of at least 5 ng/ml and at least 50% of EBs express EpCAM and CD49f mammary gland positive progenitor-cell markers. In some embodiments, at least 75% of EBs express EpCAM and CD49f mammary gland positive progenitor-cell markers. In some embodiments, BMP4 is added to the culture medium in a concentration of at least 20 ng/ml and at least 75% of EBs express EpCAM and CD49f mammary gland positive progenitor-cell markers.

In some embodiments, at least 15% of EBs express MUC1 and CD49f mammary gland positive progenitor-cell markers. In some embodiments, BMP4 is added to the culture medium in a concentration of at least 5 ng/ml and at least 15% of EBs express MUC1 and CD49f mammary gland positive progenitor-cell markers. In some embodiments, at least 20% of EBs express MUC1 and CD49f mammary gland positive progenitor-cell markers. In some embodiments, BMP4 is added to the culture medium in a concentration of at least 20 ng/ml and at least 20% of EBs express MUC1 and CD49f in mammary gland positive progenitor-cell markers.

In some embodiments, at least 20% of EBs express MUC1 and EpCAM mammary gland positive progenitor-cell markers. In some embodiments, BMP4 is added to the culture medium in a concentration of at least 5 ng/ml and at least 20% of EBs express MUC1 and EpCAM mammary gland positive progenitor-cell markers. In some embodiments, at least 35% of EBs express MUC1 and EpCAM mammary gland positive progenitor-cell markers. In some embodiments, BMP4 is added to the culture medium in a concentration of at least 20 ng/ml and at least 35% of EBs express MUC1 and EpCAM mammary gland positive progenitor-cell markers.

In some embodiments, at least 15% of EBs express GATA3 mammary gland positive progenitor-cell markers. In some embodiments, BMP4 is added to the culture medium in a concentration of at least 20 ng/mL and at least 15% of EBs express GATA3 mammary gland positive progenitor-cell markers.

The EBs also express non-neuronal ectodermal markers, thereby demonstrating enrichment towards the non-neuronal lineage. In some embodiments, said one or more non-neuronal ectodermal markers are selected from TFAP2A and TFAP2C.

In some embodiments, the EBs have increased expression of one or more non-neuronal ectodermal markers of at least 2-fold compared to the expression level of said non-neuronal ectodermal markers in EBs not treated with BMP4.

In some embodiments, the EBs have increased expression of one or more non-neuronal ectodermal markers of at least 3 to 15-fold compared to the expression level of said non-neuronal ectodermal markers in EBs not treated with BMP4.

In some embodiments, the EBs have increased expression of the non-neuronal ectodermal marker TFAP2A of at least 2-fold compared to the expression level of said non-neuronal ectodermal markers in EBs not treated with BMP4. In some embodiments, the EBs have increased expression of the non-neuronal ectodermal marker TFAP2C of at least 2-fold compared to the expression level of said non-neuronal ectodermal markers in EBs not treated with BMP4.

In some embodiments, the EBs have decreased expression of one or more neuronal ectodermal markers. In some embodiments, said one or more neuronal ectodermal markers are selected from PAX6, OTX2 and SOX11.

The methods as described anywhere herein therefore provide a more homogeneous cell population in terms of the types of cells present in the cell population, i.e., the cell population comprises a more homogenous population of non-neuronal ectodermal lineage cells.

In some embodiments, the EBs have decreased expression of one or more neuronal ectodermal markers of at least 0.5-fold compared to the expression level of said neuronal ectodermal markers in EBs not treated with BMP4, i.e., the expression level of these markers is reduced by half. In some embodiments, said neuronal ectodermal markers are selected from PAX6, OXT2 and SOX11.

In some embodiments, the EBs express one or more milk-specific bioactive markers. In some embodiments, said milk-specific bioactive marker is osteopontin (OPN).

In some embodiments, the EBs have increased expression of one or more milk-specific bioactive markers compared to the expression level of said non-neuronal ectodermal markers in EBs not treated with BMP4.

In some embodiments, the EBs have increased expression of one or more milk-specific bioactive markers of at least 2-fold compared to the expression level of said non-neuronal ectodermal markers in EBs not treated with BMP4.

In some embodiments, the EBs have increased expression of OPN of at least 2-fold compared to the expression level of said non-neuronal ectodermal markers in EBs not treated with BMP4. In some embodiments, BMP4 is added to the culture medium in a concentration of 5 to 20 ng/ml and the EBs have increased expression of OPN of at least 2-fold compared to the expression level of said non-neuronal ectodermal markers in EBs not treated with BMP4.

In some embodiments, the EBs have increased expression of OPN of at least 4-fold compared to the expression level of said non-neuronal ectodermal markers in EBs not treated with BMP4.

In some embodiments, the EBs have increased expression of OPN of at least 18-fold compared to the expression level of said non-neuronal ectodermal markers in EBs not treated with BMP4.

In some embodiments, the mammary gland cells generate increased expression of milk-specific bioactive markers compared to mammary gland cells that are not treated with BMP4. In some embodiments, the mammary gland cells generate at least a 2-fold increase in expression of milk-specific bioactive markers compared to mammary gland cells that are not treated with BMP4. In some embodiments, said markers are selected from estrogen related receptor alpha (ESRRA), Keratin 14 (KRT14) and MUC1. In some embodiments, the mammary gland cells generate at least a 2-fold increase in expression ESRRA compared to mammary gland cells that are not treated with BMP4. In some embodiments, the mammary gland cells generate at least a 2-fold increase in expression KRT14 compared to mammary gland cells that are not treated with BMP4. In some embodiments, the mammary gland cells generate at least a 2-fold increase in expression MUC1 compared to mammary gland cells that are not treated with BMP4.

### Retinoic Acid (RA)

In some embodiments, RA is added to the culture conditions in the early stages of the differentiation methods as described anywhere herein. In some embodiments, RA is added to the culture medium during the mammary lineage commitment stage. In some embodiments, RA is added to the culture medium between day 10 and day 15. In some embodiments, RA is added to the culture medium between day 6 and day 11. In some embodiments, RA is not added to the culture medium after day 11. In some embodiments, RA is added to the culture medium before day 11. Day 0 is the time point where the hBSCs are first added to the culture medium, i.e. when the hBSCs are first induced for differentiation.

In some embodiments, RA is added to the culture medium for 5 days.

In some embodiments, RA is added to the culture medium in a concentration of 1 μM.

In some embodiments, RA is added to the culture conditions between day 10 and day 15, and in a concentration of between 1 μM.

The EBs generated in the methods as described anywhere express one or more mammary gland positive progenitor-cell markers. In some embodiments, said one or more mammary gland positive progenitor-cell markers are selected from EpCAM, CD49f and MUC1.

In some embodiments, the EBs have increased expression of one or more mammary gland positive progenitor-cell markers compared to the expression level of said mammary gland positive progenitor-cell markers in EBs not treated with RA.

In some embodiments, the EBs have increased expression of one or more mammary gland positive progenitor-cell markers compared to the expression level of said mammary gland positive progenitor-cell markers in EBs treated with serum, such as Bovine Serum Albumin (BSA) or Fetal Bovine Serum.

In some embodiments, at least 10%, 20%, 30%, 40%, 50% or more of the EBs express one or more mammary gland positive progenitor-cell markers. In some embodiments, at least 10%, 20%, 30%, 40%, 50%, 60%, 70%, 80% or more of the EBs express two or more mammary gland positive progenitor-cell markers. In some embodiments, said mammary gland positive progenitor-cell markers are selected from EpCAM, CD49f and MUC1.

In some embodiments, at least 40% of EBs express EpCAM and CD49f mammary gland positive progenitor-cell markers. In some embodiments, at least 10% of EBs express MUC1 and CD49f mammary gland positive progenitor-cell markers. In some embodiments, at least 15% of EBs express MUC1 and EpCAM mammary gland positive progenitor-cell markers.

In some embodiments, the EBs have increased cell viability compared to EBs not treated with RA. In some embodiments, the EBs have at least 85% or more viability. In some embodiments, the EBs have at least 95% or more viability.

The methods of producing a population of mammary gland cells as described anywhere herein, may be combined and/or utilized in the methods for producing a mammalian milk like product as described anywhere herein, particular as part of Step A) as described anywhere herein.

For example, in some embodiments, of the method of producing a population of mammary gland cells as described anywhere herein, the culturing step i) comprises culturing the hBSCs in MammoCult medium, in a 3D-suspension culture system, for example 3D-suspension condition, thereby directing the hBSCs to differentiate towards non-neural ectoderm cells, optionally for at least 12 days.

In some embodiments in some embodiments of the method of producing a population of mammary gland cells as described anywhere herein, the growing step ii) comprises growing the EBs in a 3D embedding system comprising RA, for example a mixed floating gel composed of matrix protein such as Matrigel and/or Collagen I for at least 30 days, for example for 32 days.

In some embodiments, step ii) is distinguished into further substeps and comprises the following steps: ii) and iii):
ii) incubation of mEBs (mammospheres) in complete EpiCultB medium supplemented with EpiCult proliferation supplement, Parathyroid hormone (pTHrP), and RA for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20.

In some embodiments, step ii) is distinguished into further substeps and comprises the following steps: ii) and iii):
ii) embedding the formed mEBs (mammospheres) in a mixture of Matrigel and Collagen I floated in EpiCultB medium supplemented with EpiCult proliferation supplement, Parathyroid hormone (pTHrP), and RA for 5 days,
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days.

In some embodiments of the method of producing a population of mammary gland cells as described anywhere herein, the culturing step i) comprises culturing the hBSCs in MammoCult medium, in a 3D-suspension culture system, for example 3D-suspension condition, thereby directing the hBSCs to differentiate towards non-neural ectoderm cells, optionally for at least 12 days, and the growing step ii) comprises growing the EBs in a 3D embedding system comprising RA, for example a mixed floating gel composed of matrix protein such as Matrigel and/or Collagen I for at least 30 days, for example for 32 days.

### BMP4 and RA

It will be understood that any embodiment relating to BMP4 as described anywhere herein may be combined with any embodiment relating RA as described anywhere herein.

For example, in relation to the timings for adding BMP4 and RA to the culture medium, in some embodiments, BMP4 is added to the culture medium between day 0 and day 3, and RA is added to the culture medium between day 10 and day 15.

In some embodiments, BMP4 is added to the culture medium between day 0 and day 3, and RA is added to the culture medium between day 6 and day 11.

It will also be understood that the combination of BMP4 and RA in the methods as described anywhere herein demonstrates additional surprising effects, beyond what is observed for each of BMP4 and RA alone.

The EBs generated in the methods using a combination of BMP4 and RA as described anywhere herein express one or more mammary gland positive progenitor-cell markers. In some embodiments, said one or more mammary gland positive progenitor-cell markers are selected from EpCAM, CD49f, MUC1 and GATA3.

In some embodiments, the EBs have increased expression of one or more mammary gland positive progenitor-cell markers compared to the expression level of said mammary gland positive progenitor-cell markers in EBs not treated with BMP4.

In some embodiments, at least 10%, 20%, 30%, 40%, 50%, 60%, 70%, 80% or more of the EBs express one or more mammary gland positive progenitor-cell markers. In some embodiments, at least 10%, 20%, 30%, 40%, 50%, 60%, 70%, 80% or more of the EBs express two or more mammary gland positive progenitor-cell markers. In some embodiments, said mammary gland positive progenitor-cell markers are selected from EpCAM, CD49f, MUC1 and GATA3.

In some embodiments, at least 35% of EBs express EpCAM and CD49f mammary gland positive progenitor-cell markers. In some embodiments, at least 60% of EBs express EpCAM and CD49f mammary gland positive progenitor-cell markers at the middle differentiation stage. In some embodiments, at least 35% of EBs express EpCAM and CD49f mammary gland positive progenitor-cell markers at the pre-induction stage. In some embodiments, at least 15% of EBs express EpCAM and CD49f mammary gland positive progenitor-cell markers at the post-induction stage. In some embodiments, at least 40% of EBs express EpCAM and CD49f mammary gland positive progenitor-cell markers at the post-induction stage.

In some embodiments, at least 15% of EBs express MUC1 and EpCAM mammary gland positive progenitor-cell markers. In some embodiments, at least 20% of EBs express MUC1 and EpCAM mammary gland positive progenitor-cell markers at the middle differentiation stage. In some embodiments, at least 15% of EBs express MUC1 and EpCAM mammary gland positive progenitor-cell markers at the pre-induction stage. In some embodiments, at least 5% of EBs express MUC1 and EpCAM mammary gland positive progenitor-cell markers at the post-induction stage. In some embodiments, at least 15% of EBs express MUC1 and EpCAM mammary gland positive progenitor-cell markers at the post-induction stage.

In some embodiments, at least 20% of EBs express GATA3 and EpCAM mammary gland positive progenitor-cell markers. In some embodiments, at least 50% of EBs express GATA3 and EpCAM mammary gland positive progenitor-cell markers at the middle differentiation stage. In some embodiments, at least 25% of EBs express GATA3 and EpCAM mammary gland positive progenitor-cell markers at the pre-induction stage. In some embodiments, at least 15% of EBs express GATA3 and EpCAM mammary gland positive progenitor-cell markers at the post-induction stage. In some embodiments, at least 40% of EBs express GATA3 and EpCAM mammary gland positive progenitor-cell markers at the post-induction stage.

In some embodiments, the middle differentiation stage is day 25, the pre-induction stage is day 35 and the post-induction stage is day 42. In some embodiments, the middle differentiation stage is between day 20. The pre-induction stage is day 26 and the post-induction stage is day 31.

In some embodiments, the EBs express one or more milk-specific bioactive markers. In some embodiments, said milk-specific bioactive marker is osteopontin (OPN).

In some embodiments, the EBs have increased expression of one or more milk-specific bioactive markers compared to the expression level of said markers in EBs not treated with BMP4 and RA. In some embodiments, the EBs have increased secretion of one or more milk-specific bioactive markers compared to the expression level of said markers in EBs not treated with BMP4 and RA. In some embodiments, the EBs have increased secretion of OPN compared to the expression level of OPN in EBs not treated with BMP4 and RA. In some embodiments, the EBs have 40% or more increased secretion of OPN compared to the expression level of OPN in EBs not treated with BMP4 and RA.

The methods of producing a population of mammary gland cells as described anywhere herein, may be combined and/or utilized in the methods for producing a mammalian milk like product as described anywhere herein, particular as part of Step A) as described anywhere herein.

For example, in some embodiments of the method of producing a population of mammary gland cells as described anywhere herein, the culturing step i) comprises culturing the hBSCs in MammoCult medium and optionally BMP4, in a 3D-suspension culture system, for example 3D-suspension condition, thereby directing the hBSCs to differentiate towards non-neural ectoderm cells, optionally for at least 12 days. In some embodiments, step A i) is for no more than 8 days.

In some embodiments in some embodiments of the method of producing a population of mammary gland cells as described anywhere herein, the growing step ii) comprises growing the formed EBs in a 3D embedding system optionally comprising RA, for example a mixed floating gel composed of matrix protein such as Matrigel and/or Collagen I for at least 30 days, for example for 32 days, to generate lactocytes. In some embodiments, step A ii) is for at least 23 days, for example no more than 25 days.

In some embodiments of the method of producing a population of mammary gland cells as described anywhere herein, the culturing step i) comprises culturing the hBSCs in MammoCult medium and BMP4, in a 3D-suspension culture system, for example 3D-suspension condition, thereby directing the hBSCs to differentiate towards non-neural ectoderm cells, optionally for at least 12 days, and the growing step ii) comprises growing the formed EBs in a 3D embedding system comprising RA, for example a mixed floating gel composed of matrix protein such as Matrigel and/or Collagen I for at least 30 days, for example for 32 days, to generate lactocytes.

### Mammalian Milk Like Product Production

The present invention also relates to methods for producing a mammalian milk like product as defined herein, including any of steps A) and B) as defined herein and optional step C) as defined herein. Said methods for producing a mammalian milk like product as defined herein may also include the methods of producing mammalian mammary cells, as part of step A). In particular, BMP4 and/or RA may be added to any of the methods for producing a mammalian milk like product as defined herein, particularly as part of step A). Said methods using a combination of BMP4 and RA also include different lengths of time for producing the mammalian milk like product.

Step A—Generating Lactocytes and/or Mammary Like Organoids from hBSCs

According to the method of the present invention, mammary like cells and/or organoid structures are generated under step A).

Such mammary like cells and/or organoid structures can be generated according to any reported method making use of hBSC.

In one embodiment, such mammary like cells and/or organoid structures may be generated according to the procedure described in Hassiotou F. et al. Stem Cells. 2012 which is hereby incorporated in its entirety.

The methodology described in the above mentioned scientific publication (herebelow also referred to as “Hassiotou publication”) represents a protocol to generate human mammary like cells and/or organoids from hBSCs.

More specifically, step A preferably includes:
Embryoid bodies formation
Mammary lineage commitment
Branch and alveolar differentiation, and
Induction of milk bioactives.

Each of these stages may be conducted for a specific amount of time, with specific culture mediums.

In some embodiments, step A is conducted for a total of 40 to 45 days, preferably 42 days.

In some embodiments, step A is shortened and is conducted for less than 42 days, optionally less than 40 days, optionally less than 31 days. Preferably step A is conducted for 31 days.

In some embodiments, the embryoid bodies formation stage is between day 0 and day 10. In some embodiments, the embryoid bodies formation stage is for 10 days.

In some embodiments, the embryoid bodies formation stage is shortened and is between day 0 and day 6. In some embodiments, the embryoid bodies formation stage is for 6 days. In some embodiments, the embryoid bodies formation stage is for 10 days or less.

In some embodiments, the mammary lineage commitment stage is between day 10 and day 15. In some embodiments, the mammary lineage commitment stage is between day 6 and day 11. In some embodiments, the embryoid bodies formation stage is for 5 days. In some embodiments, the embryoid bodies formation stage is for no more than 5 days.

In some embodiments, the branch and alveolar differentiation stage is between day 15 and day 35. In some embodiments, the branch and alveolar differentiation stage is for 20 days.

In some embodiments, the branch and alveolar differentiation stage is shortened and is between day 11 and day 26. In some embodiments, the branch and alveolar differentiation stage is for 15 days. In some embodiments, the branch and alveolar differentiation stage is for no more than 15 days.

In some embodiments, the induction of milk bioactive stage is between day 35 and day 42. In some embodiments, the induction of milk bioactive stage is for 7 days. In some embodiments, the induction of milk bioactive stage is shortened and is between day 26 and day 31. In some embodiments, the induction of milk bioactive stage is for 5 days. In some embodiments, the induction of milk bioactive stage is for no more than 5 days.

Thus, in some embodiments, step A is conducted for a total of 42 days, wherein the embryoid bodies formation stage is between day 0 and day 10, the mammary lineage commitment stage is between day 10 and day 15, the branch and alveolar differentiation stage is between day 15 and day 35, and the induction of milk bioactive stage is between day 35 and day 42.

In other embodiments, the process is shortened such that step A is conducted for a total of 31 days, wherein the embryoid bodies formation stage is between day 0 and day 6, the mammary lineage commitment stage is between day 6 and day 11, the branch and alveolar differentiation stage is between day 11 and day 26, and the induction of milk bioactive stage is day 26 and day 31.

Further details of the time periods and culture mediums for use in the methods described anywhere herein are provided below.

In some embodiments, BMP4 is added to the culture medium. This is to increase the proportion of mammary gland progenitor cells as described herein.

BMP4 is added to the culture medium as described in anywhere herein.

BMP4 is added to the culture conditions in the early stages of the differentiation methods as described anywhere herein. In some embodiments, BMP4 is added to the culture medium between day 0 and day 10. In some embodiments, BMP4 is added to the culture medium between day 0 and day 6. In some embodiments, BMP4 is added to the culture medium between day 0 and day 3. Day 0 is the time point where the hBSCs are first added to the culture medium, i.e. when the hBSCs are first induced for differentiation.

In some embodiments, BMP4 is added to the culture medium for 3 days.

In some embodiments, BMP4 is added to the culture medium in a concentration of 5 to 20 ng/ml. In some embodiments, BMP4 is added to the culture medium in a concentration of 5 ng/mL. In some embodiments, BMP4 is added to the culture medium in a concentration of 10 ng/ml. In some embodiments, BMP4 is added to the culture medium in a concentration of 20 ng/mL.

In some embodiments, BMP4 is added to the culture medium between day 0 and day 3, and in a concentration of between 5 to 20 ng/ml.

In some embodiments, RA is added to the culture medium in step 2. This is to increase the proportion of mammary gland progenitor cells and/or increase the viability of mammary gland progenitor cells.

RA is added to the culture medium as described in anywhere herein.

RA is added to the culture medium in the early stages of the differentiation methods as described anywhere herein. In some embodiments, RA is added to the culture medium during the mammary lineage commitment stage. In some embodiments, RA is added to the culture medium between day 10 and day 15. In some embodiments, RA is added to the culture medium between day 6 and day 11. In some embodiments, RA is not added to the culture medium after day 11. Day 0 is the time point where the hBSCs are first added to the culture medium, i.e. when the hBSCs are first induced for differentiation.

In some embodiments, RA is added to the culture medium for 5 days.

In some embodiments, RA is added to the culture medium in a concentration of 1 μM.

In some embodiments, RA is added to the culture medium between day 10 and day 15, and in a concentration of between 1 μM.

In one embodiment of the present invention, a method for producing a human milk like product is provided comprising generating lactocytes under step a) from human adult breast milk stem cells (hBSCs), where such step a) comprises:
i) culturing hBSCs in an appropriate culture medium (for example MammoCult medium, optionally supplemented with antibiotic-antimicotic solution and fungizone) and after one week collecting mammospheres formed thereof; and
ii) growing such mammospheres in an appropriate system (such as a mammary differentiation medium comprising for example culture medium RPMI (Roswell Park Memorial Institute) 1640 with L-glutamine optionally supplemented with fetal bovine serum (FBS), insulin, epidermal growth factors (EGF), hydrocortisone, antibiotic-antimicotic solution and fungizone) for at least 1 week, for example 2 to 4 weeks, to generate lactocytes.

In one embodiment of the present invention, a method for producing a human milk like product is provided comprising generating lactocytes under step a) from human adult breast milk stem cells (hBSCs), where such step a) comprises:
i) culturing hBSCs in an appropriate culture medium (for example MammoCult medium, optionally supplemented with antibiotic-antimicotic solution and fungizone) optionally comprising BMP4 as described anywhere herein, and after one week collecting mammospheres formed thereof; and
ii) growing such mammospheres in an appropriate system optionally comprising RA as described anywhere herein (such as a mammary differentiation medium comprising for example culture medium RPMI (Roswell Park Memorial Institute) 1640 with L-glutamine optionally supplemented with fetal bovine serum (FBS), insulin, epidermal growth factors (EGF), hydrocortisone, antibiotic-antimicotic solution and fungizone) for at least 1 week, for example 2 to 4 weeks, to generate lactocytes.

In one embodiment of the present invention, a method for producing a human milk like product is provided comprising generating lactocytes under step a) from human adult breast milk stem cells (hBSCs), where such step a) comprises:
i) culturing hBSCs in an appropriate culture medium (for example MammoCult medium, optionally supplemented with antibiotic-antimicotic solution and fungizone) comprising BMP4 as described anywhere herein, and after 6 days collecting mammospheres formed thereof; and
ii) growing such mammospheres in an appropriate system comprising RA as described anywhere herein (such as a mammary differentiation medium comprising for example culture medium RPMI (Roswell Park Memorial Institute) 1640 with L-glutamine optionally supplemented with fetal bovine serum (FBS), insulin, epidermal growth factors (EGF), hydrocortisone, antibiotic-antimicotic solution and fungizone) for less than 10 days, to generate lactocytes.

In one embodiment of the present invention, a method for producing a human milk like product is provided comprising generating lactocytes under step a) from human adult breast milk stem cells (hBSCs), where such step a) comprises:
i) aggregating and culturing hBSCs in an appropriate culture medium (for example MammoCult medium) in non-adherent conditions for mammospheres formation; and
ii) growing such mammospheres in a 3D appropriate system (for example a mixed floating gel composed of matrix protein such as Matrigel and/or Collagen or in suspension cultures in non-adherent plates) for at least 10 days to generate lactocytes.

In one embodiment of the present invention, a method for producing a human milk like product is provided comprising generating lactocytes under step a) from human adult breast milk stem cells (hBSCs), where such step a) comprises:
i) aggregating and culturing hBSCs in an appropriate culture medium (for example MammoCult medium) optionally comprising BMP4 as described anywhere herein, in non-adherent conditions for mammospheres formation; and
ii) growing such mammospheres in a 3D appropriate system optionally comprising RA as described anywhere herein (for example a mixed floating gel composed of matrix protein such as Matrigel and/or Collagen or in suspension cultures in non-adherent plates) for at least 10 days to generate lactocytes.

In one embodiment of the present invention, a method for producing a human milk like product is provided comprising generating lactocytes under step a) from human adult breast milk stem cells (hBSCs), where such step a) comprises:
i) aggregating and culturing hBSCs in an appropriate culture medium (for example MammoCult medium) comprising BMP4 as described anywhere herein, in non-adherent conditions for mammospheres formation; and
ii) growing such mammospheres in a 3D appropriate system comprising RA as described anywhere herein (for example a mixed floating gel composed of matrix protein such as Matrigel and/or Collagen or in suspension cultures in non-adherent plates) for less than 10 days to generate lactocytes.

In one embodiment, mammary commitment under step A) is obtained by applying a conditioned medium (for example EpiCultB) supplemented with specific factors (for example Parathyroid hormone (pTHrP), hydrocortisone, insulin, FGF10, and HGF).

In one embodiment, mammary commitment under step A) is obtained by applying a conditioned medium (for example EpiCultB) supplemented with specific factors (for example Parathyroid hormone (pTHrP), hydrocortisone, insulin, FGF10, and HGF) and RA.

In one embodiment of the present invention, the method comprises generating mammary-like organoids under step A).

In one embodiment of the present invention, the method to generate mammary-like organoids under step A) includes culturing the cells under conditions selected from the group consisting of: 2D monolayers of cells, 2D with attached EBs, in suspension in non-adherent plates and in mixed floating gel.

In another preferred embodiment, mammospheres (mEBs) in step A) are grown in an appropriate system (for example a floating mixed gel culture system as described in Hassiotou F. et al. Stem Cells. 2012) for at least 15 days.

In a more preferred embodiment, mammospheres (mEBs) in step A) are grown in an appropriate system (for example a floating mixed gel culture system as described in Hassiotou F. et al. Stem Cells. 2012) for 20 days.

In one embodiment, the method according to the present invention provides for culture conditions according to step A) [for example under step A)i) and/or under step A)ii)] which are adapted to generate lactocytes derived from human adult breast milk stem cells (hBSCs) capable to secrete a standard human milk like product.

In one embodiment, delivery of nutrients and biomimetic stimuli is controlled to influence cell growth, differentiation and tissue formation. In one embodiment, such control is performed in a bioreactor.

In one embodiment, the method according to the present invention provides for culture conditions according to step A) [for example under step A)i) and/or under step A)ii)] which are adapted to generate lactocytes derived from human adult breast milk stem cells (hBSCs) capable to secrete a non-standard human milk like product.

In a preferred embodiment, a method for producing a human milk like product is provided comprising generating lactocytes under step A) from human hBSC where such step A) comprises directing hBSCs to differentiate towards mammary gland cells (for example lactocytes) in an appropriate 3D culture system (for example 3D-suspension condition) for at least 42 days. In some embodiments, for at least 31 days. In some embodiments, for not more than 31 days.

In a preferred embodiment, a method for producing a human milk like product is provided comprising generating lactocytes under step A) from human hBSC where such step A) comprises directing hBSCs to differentiate towards mammary gland cells (for example lactocytes) in an appropriate 3D culture system comprising BMP4 and/or RA as described anywhere herein (for example 3D-suspension condition) for at least 42 days. In a preferred embodiment, a method for producing a human milk like product is provided comprising generating lactocytes under step A) from human hBSC where such step A) comprises directing hBSCs to differentiate towards mammary gland cells (for example lactocytes) in an appropriate 3D culture system comprising BMP4 and RA as described anywhere herein (for example 3D-suspension condition) for no more than 31 days.

In another preferred embodiment, a method for producing a human milk like product is provided comprising generating lactocytes under step A) from hBSC where such step A) comprises:
i) directing hBSC to differentiate towards non-neural ectoderm cells by culturing them in an appropriate culture medium (for example MammoCult medium) in an appropriate 3D culture system (for example 3D-suspension condition) for at least 12 days (day-2 to day 10), and
ii) growing the formed mEBs (mammospheres) in an appropriate 3D embedding system (for example a mixed floating gel composed of matrix protein such as Matrigel and/or Collagen I) for at least 30 days, preferably for 32 days, to generate lactocytes.

In another preferred embodiment, a method for producing a human milk like product is provided comprising generating lactocytes under step A) from hBSC where such step A) comprises:

i) directing hBSC to differentiate towards non-neural ectoderm cells by culturing them in an appropriate culture medium (for example MammoCult medium) optionally comprising BMP4 as described anywhere herein, in an appropriate 3D culture system (for example 3D-suspension condition) for at least 12 days (day-2 to day 10), and
ii) growing the formed mEBs (mammospheres) in an appropriate 3D embedding system optionally comprising RA (for example a mixed floating gel composed of matrix protein such as Matrigel and/or Collagen I) for at least 30 days, preferably for 32 days, to generate lactocytes.

In another preferred embodiment, a method for producing a human milk like product is provided comprising generating lactocytes under step A) from hBSC where such step A) comprises:
i) directing hBSC to differentiate towards non-neural ectoderm cells by culturing them in an appropriate culture medium (for example MammoCult medium) comprising BMP4 as described anywhere herein, in an appropriate 3D culture system (for example 3D-suspension condition) for no more than 8 days (day-2 to day 6), and
ii) growing the formed mEBs (mammospheres) in an appropriate 3D embedding system comprising RA (for example a mixed floating gel composed of matrix protein such as Matrigel and/or Collagen I) for no more than 25 days, to generate lactocytes.

In a particularly preferred embodiment of the present invention, a method of producing a human milk like product is provided comprising generating lactocytes under step A) from hBSCs, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO₃ and transferrin, TGFβ1 or NODAL as described in Chen et al., Nat Methods, 2011) or mTeSR™ for two days (day-2-day 0), and producing mEBs (mammospheres) highly enriched in non-neural ectodermal cells by incubation of EBs in complete MammoCult medium (StemCell Technologies) comprising the basal medium, proliferation supplement and supplemented with heparin (typically 4 μg/mL), and hydrocortisone (typically 0.48 μg/mL) for 10 days (day 0-day 10), and wherein step A)ii) is distinguished into further substeps and comprises the following steps: ii), iii) and iv):
ii) incubation of mEBs (mammospheres) in complete EpiCultB medium supplemented with EpiCult proliferation supplement and Parathyroid hormone (pTHrP) for 5 days (day 10-day 15),
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days (day 15-day 35), and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days (day 35-day 42).

In a particularly preferred embodiment of the present invention, a method of producing a human milk like product is provided comprising generating lactocytes under step A) from hBSCs, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO₃ and transferrin, TGFβ1 or NODAL as described in Chen et al., Nat Methods, 2011) or mTeSR™ for two days (day-2-day 0), and producing mEBs (mammospheres) highly enriched in non-neural ectodermal cells by incubation of EBs in complete MammoCult medium (StemCell Technologies) comprising the basal medium, proliferation supplement and supplemented with heparin (typically 4 μg/mL), hydrocortisone (typically 0.48 μg/mL) and optionally BMP4 as described anywhere herein, for 10 days (day 0-day 10), and wherein
step A)ii) is distinguished into further substeps and comprises the following steps: ii), iii) and iv):

ii) incubation of mEBs (mammospheres) in complete EpiCultB medium supplemented with EpiCult proliferation supplement and Parathyroid hormone (pTHrP) and optionally RA as described anywhere herein, for 5 days (day 10-day 15),
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days (day 15-day 35), and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days (day 35-day 42).

In a particularly preferred embodiment of the present invention, a method of producing a human milk like product is provided comprising generating lactocytes under step A) from hBSCs, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO₃ and transferrin, TGFβ1 or NODAL as described in Chen et al., Nat Methods, 2011) or mTeSR™ for two days (day-2-day 0), and producing mEBs (mammospheres) highly enriched in non-neural ectodermal cells by incubation of EBs in complete MammoCult medium (StemCell Technologies) comprising the basal medium, proliferation supplement and supplemented with heparin (typically 4 μg/mL), hydrocortisone (typically 0.48 μg/mL) and BMP4 as described anywhere herein, for 6 days (day 0-day 6), and wherein step A)ii) is distinguished into further substeps and comprises the following steps: ii), iii) and iv):
ii) incubation of mEBs (mammospheres) in complete EpiCultB medium supplemented with EpiCult proliferation supplement and Parathyroid hormone (pTHrP) and RA as described anywhere herein, for 5 days (day 6-day 11),
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 15 days (day 11-day 26), and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 5 days (day 26-day 31).

Step iv) preferably leads to differentiation into milk protein expressing cells, particularly lactocytes, and/or mammary like gland organoids.

In a further particularly preferred embodiment of the present invention, a method of producing a human milk like product is provided comprising generating lactocytes under step A) from hBSC, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO₃ and transferrin, TGFβ1 or NODAL as described in Chen et al., Nat Methods, 2011) or mTeSR™ for two days (day-2-day 0), and producing mEBs (mammospheres) highly enriched in non-neural ectodermal cells by incubation of EBs in MammoCultB medium supplemented with MammoCult proliferation supplement, hydrocortisone and heparin for 10 days (day 0-day 10), and wherein step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement and Parathyroid hormone (pTHrP) for 5 days (day 10-day 15),
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days (day 15 to day 35), and
iv) induction of milk protein expression by incubating mEBs (mammospheres) in EpiCultB medium supplemented with EpiCult t proliferation supplement, hydrocortisone, insulin, FBS, prolactin, progesterone and β-estradiol for 7 days (day 35 to day 42).

In a further particularly preferred embodiment of the present invention, a method of producing a human milk like product is provided comprising generating lactocytes under step A) from hBSC, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO₃ and transferrin, TGFβ1 or NODAL as described in Chen et al., Nat Methods, 2011) or mTeSR™ for two days (day-2-day 0), and producing mEBs (mammospheres) highly enriched in non-neural ectodermal cells by incubation of EBs in MammoCultB medium supplemented with MammoCult proliferation supplement, hydrocortisone, heparin and optionally BMP4 as described anywhere herein, for 10 days (day 0-day 10), and wherein step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement and Parathyroid hormone (pTHrP) and optionally RA for 5 days (day 10-day 15),
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days (day 15 to day 35), and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days (day 35 to day 42).

In a further particularly preferred embodiment of the present invention, a method of producing a human milk like product is provided comprising generating lactocytes under step A) from hBSC, wherein step A)i) is defined as follows:
i) generation of embryoid bodies (EBs) from hBSC by incubation in standard medium E8 (comprising DMEM/F12, L-ascorbic acid-2-phosphate magnesium, sodium selenium, FGF2, insulin, NaHCO₃ and transferrin, TGFβ1 or NODAL as described in Chen et al., Nat Methods, 2011) or mTeSR™ for two days (day-2-day 0), and producing mEBs (mammospheres) highly enriched in non-neural ectodermal cells by incubation of EBs in MammoCultB medium supplemented with MammoCult proliferation supplement, hydrocortisone, heparin and BMP4 as described anywhere herein, for 6 days (day 0-day 6), and wherein step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement and Parathyroid hormone (pTHrP) and RA for 5 days (day 6-day 11),
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 15 days (day 11 to day 26), and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 5 days (day 26 to day 31).

Step iv) preferably leads to differentiation into milk protein expressing cells, particularly lactocytes, and/or mammary like gland organoids.

Standard medium E8 (comprising DMEM/F12, L-ascorbic acid-2-phosphate magnesium, sodium selenium, FGF2, insulin, NaHCO₃ and transferrin, TGFβ1 or NODAL as described in Chen et al., Nat Methods, 2011) as mentioned herein is commercially available, e.g., as “Essential 8™ Medium” from ThermoFischer Scientific, catalogue number A1517001 (see also https://www.thermofisher.com/order/catalog/product/A1517001 #/A1517001).

mTeSR™ medium is commercially available from STEMCELL Technologies, catalogue number 85850 (see also https://www.stemcell.com/mtesr1.html). Suchc medium is also described in “Defined, Feeder-Independent medium for human hembryonic stem cell culture”, Current protocol in Stem Cell Biology, Volume 2, Issue 1, September 2007. In one embodiment, steps iii) and/or iv) as defined above for the particularly preferred embodiments, preferably lead to formation of/differentiation into at least breast cells, luminal cells, and basal cells. In this context, breast cells preferably express one or more, preferably all of markers selected from the group consisting of: β-Casein, milk protein, and hormone receptors. Moreover, luminal cells preferably express one or more, preferably all markers selected from the group consisting of: EpCAM, MUC1, CD49F, GATA3, CK8, and CK18. Moreover, basal cells preferably express one or more markers selected from the group consisting of: CK14, α-smooth muscle actin and P63.

In one further embodiment, after induction of mEBs (mammospheres) in step ii) and/or iv) as defined above for the particularly preferred embodiments, mammary like gland organoids may be obtained, that express one or more markers selected from the group consisting of: β-Casein, milk protein, and hormone receptors, luminal cells that express one or more markers selected from the group consisting of: EpCAM, MUC1, CD49F, GATA3, CK8, CK18, and basal cells that express one or more markers selected from the group consisting of: CK14, α-smooth muscle actin and P63.

In one embodiment of the invention, the methods above described are provided for producing a standard human milk like product.

In another embodiment, the methods above described are provided for producing a non-standard human milk like product.

In one embodiment, delivery of nutrients and biomimetic stimuli is controlled to influence cell growth, differentiation and tissue formation. In one embodiment, such control is performed in a bioreactor.

Where BMP4 is added to the culture medium in the methods for producing a mammalian milk like product as described anywhere herein, the EBs generated in the methods as described anywhere herein express one or more mammary gland positive progenitor-cell markers. In some embodiments, said one or more mammary gland positive progenitor-cell markers are selected from EpCAM, CD49f, MUC1 and GATA3.

In some embodiments, the EBs have increased expression of one or more mammary gland positive progenitor-cell markers compared to the expression level of said mammary gland positive progenitor-cell markers in EBs not treated with BMP4.

In some embodiments, at least 10%, 20%, 30%, 40%, 50%, 60%, 70%, 80% or more of the EBs express one or more mammary gland positive progenitor-cell markers. In some embodiments, at least 10%, 20%, 30%, 40%, 50%, 60%, 70%, 80% or more of the EBs express two or more mammary gland positive progenitor-cell markers. In some embodiments, said mammary gland positive progenitor-cell markers are selected from EpCAM, CD49f, MUC1 and GATA3.

In some embodiments, at least 50% of EBs express EpCAM and CD49f mammary gland positive progenitor-cell markers. In some embodiments, BMP4 is added to the culture medium in a concentration of at least 5 ng/ml and at least 50% of EBs express EpCAM and CD49f mammary gland positive progenitor-cell markers. In some embodiments, at least 75% of EBs express EpCAM and CD49f mammary gland positive progenitor-cell markers. In some embodiments, BMP4 is added to the culture medium in a concentration of at least 20 ng/ml and at least 75% of EBs express EpCAM and CD49f mammary gland positive progenitor-cell markers.

In some embodiments, at least 15% of EBs express MUC1 and CD49f mammary gland positive progenitor-cell markers. In some embodiments, BMP4 is added to the culture medium in a concentration of at least 5 ng/ml and at least 15% of EBs express MUC1 and CD49f mammary gland positive progenitor-cell markers. In some embodiments, at least 20% of EBs express MUC1 and CD49f mammary gland positive progenitor-cell markers. In some embodiments, BMP4 is added to the culture medium in a concentration of at least 20 ng/ml and at least 20% of EBs express MUC1 and CD49f mammary gland positive progenitor-cell markers.

In some embodiments, at least 20% of EBs express MUC1 and EpCAM mammary gland positive progenitor-cell markers. In some embodiments, BMP4 is added to the culture medium in a concentration of at least 5 ng/ml and at least 20% of EBs express MUC1 and EpCAM mammary gland positive progenitor-cell markers. In some embodiments, at least 35% of EBs express MUC1 and EpCAM mammary gland positive progenitor-cell markers. In some embodiments, BMP4 is added to the culture medium in a concentration of at least 20 ng/ml and at least 35% of EBs express MUC1 and EpCAM mammary gland positive progenitor-cell markers.

In some embodiments, at least 15% of EBs express GATA3 mammary gland positive progenitor-cell markers. In some embodiments, BMP4 is added to the culture medium in a concentration of at least 20 ng/ml and at least 15% of EBs express GATA3 mammary gland positive progenitor-cell markers.

The EBs also express non-neuronal ectodermal markers, thereby demonstrating enrichment towards the non-neuronal lineage. In some embodiments, said one or more non-neuronal ectodermal markers are selected from TFAP2A and TFAP2C.

In some embodiments, the EBs have increased expression of one or more non-neuronal ectodermal markers of at least 2-fold compared to the expression level of said non-neuronal ectodermal markers in EBs not treated with BMP4.

In some embodiments, the EBs have increased expression of one or more non-neuronal ectodermal markers of at least 3 to 15-fold compared to the expression level of said non-neuronal ectodermal markers in EBs not treated with BMP4.

In some embodiments, the EBs have increased expression of the non-neuronal ectodermal marker TFAP2A of at least 2-fold compared to the expression level of said non-neuronal ectodermal markers in EBs not treated with BMP4. In some embodiments, the EBs have increased expression of the non-neuronal ectodermal marker TFAP2C of at least 2-fold compared to the expression level of said non-neuronal ectodermal markers in EBs not treated with BMP4.

In some embodiments, the EBs have decreased expression of one or more neuronal ectodermal markers. In some embodiments, said one or more neuronal ectodermal markers are selected from PAX6, OTX2 and SOX11.

The methods as described anywhere herein therefore provide a more homogeneous cell population in terms of the types of cells present in the cell population, i.e., the cell population comprises a more homogenous population of non-neuronal ectodermal lineage cells.

In some embodiments, the EBs have decreased expression of one or more neuronal ectodermal markers of at least 0.5-fold compared to the expression level of said neuronal ectodermal markers in EBs not treated with BMP4, i.e., the expression level of these markers is reduced by half. In some embodiments, said neuronal ectodermal markers are selected from PAX6, OXT2 and SOX11.

In some embodiments, the EBs express one or more milk-specific bioactive markers. In some embodiments, said milk-specific bioactive marker is osteopontin (OPN).

In some embodiments, the EBs have increased expression of one or more milk-specific bioactive markers compared to the expression level of said non-neuronal ectodermal markers in EBs not treated with BMP4.

In some embodiments, the EBs have increased expression of one or more milk-specific bioactive markers of at least 2-fold compared to the expression level of said non-neuronal ectodermal markers in EBs not treated with BMP4.

In some embodiments, the EBs have increased expression of OPN of at least 2-fold compared to the expression level of said non-neuronal ectodermal markers in EBs not treated with BMP4. In some embodiments, BMP4 is added to the culture medium in a concentration of 5 to 20 ng/ml and the EBs have increased expression of OPN of at least 2-fold compared to the expression level of said non-neuronal ectodermal markers in EBs not treated with BMP4.

In some embodiments, the EBs have increased expression of OPN of at least 4-fold compared to the expression level of said non-neuronal ectodermal markers in EBs not treated with BMP4.

In some embodiments, the EBs have increased expression of OPN of at least 18-fold compared to the expression level of said non-neuronal ectodermal markers in EBs not treated with BMP4.

In some embodiments, the mammary gland cells generate increased expression of milk-specific bioactive markers compared to mammary gland cells that are not treated with BMP4. In some embodiments, the mammary gland cells generate at least a 2-fold increase in expression of milk-specific bioactive markers compared to mammary gland cells that are not treated with BMP4. In some embodiments, said markers are selected from estrogen related receptor alpha (ESRRA), Keratin 14 (KRT14) and MUC1. In some embodiments, the mammary gland cells generate at least a 2-fold increase in expression ESRRA compared to mammary gland cells that are not treated with BMP4. In some embodiments, the mammary gland cells generate at least a 2-fold increase in expression KRT14 compared to mammary gland cells that are not treated with BMP4. In some embodiments, the mammary gland cells generate at least a 2-fold increase in expression MUC1 compared to mammary gland cells that are not treated with BMP4.

Thus, as an example, provided herein is a method for producing a human milk like product, comprising generating lactocytes under step A) from human breast milk stem cells (hBSC), where such step A) comprises:
i) directing hBSCs to differentiate towards non-neural ectoderm cells by culturing them in an appropriate culture medium (for example MammoCult medium) and BMP4 as described anywhere herein, and after 10 days collecting mammospheres formed thereof; and
ii) growing such mammospheres in an appropriate system (for example a floating mixed gel culture system as described in Hassiotou F. et al. Stem Cells. 2012) for at least 10 days to generate lactocytes,
wherein the mammospheres (EBs) have increased expression of one or more mammary gland positive progenitor-cell markers compared to the expression level of said mammary gland positive progenitor-cell markers in mammospheres (EBs) not treated with BMP4.

Where RA is added to the culture medium in the methods for producing a mammalian milk like product as described anywhere herein, the EBs generated in the methods as described anywhere herein express one or more mammary gland positive progenitor-cell markers. In some embodiments, said one or more mammary gland positive progenitor-cell markers are selected from EpCAM, CD49f and MUC1.

In some embodiments, the EBs have increased expression of one or more mammary gland positive progenitor-cell markers compared to the expression level of said mammary gland positive progenitor-cell markers in EBs not treated with RA.

In some embodiments, the EBs have increased expression of one or more mammary gland positive progenitor-cell markers compared to the expression level of said mammary gland positive progenitor-cell markers in EBs treated with serum, such as Bovine Serum Albumin (BSA) or Fetal Bovine Serum.

In some embodiments, at least 10%, 20%, 30%, 40%, 50% or more of the EBs express one or more mammary gland positive progenitor-cell markers. In some embodiments, at least 10%, 20%, 30%, 40%, 50%, 60%, 70%, 80% or more of the EBs express two or more mammary gland positive progenitor-cell markers. In some embodiments, said mammary gland positive progenitor-cell markers are selected from EpCAM, CD49f and MUC1.

In some embodiments, at least 40% of EBs express EpCAM and CD49f mammary gland positive progenitor-cell markers. In some embodiments, at least 10% of EBs express MUC1 and CD49f mammary gland positive progenitor-cell markers. In some embodiments, at least 15% of EBs express MUC1 and EpCAM mammary gland positive progenitor-cell markers.

In some embodiments, the EBs have increased cell viability compared to EBs not treated with RA. In some embodiments, the EBs have at least 85% or more viability. In some embodiments, the EBs have at least 95% or more viability.

Thus, as an example, provided herein is a method for producing a human milk like product in vitro comprising:
A) Generating lactocyte mammary-like gland organoids derived from human breast milk stem cells (hBSC);
B) Secreting the human milk like product from said lactocytes;
   wherein such step A) comprises:
   i) directing hBSCs to differentiate towards non-neural ectoderm cells by culturing them in an appropriate culture medium, for example MammoCult medium, in an appropriate 3D culture system, for example 3D-suspension condition, for at least 12 days and
   ii) growing the formed mEBs (mammospheres) in an appropriate 3D embedding system comprising RA, for example a mixed floating gel composed of matrix protein such as Matrigel and/or Collagen I and RA for at least 30 days, for example for 32 days, to generate lactocytes
   wherein the mammospheres (EBs) have increased expression of one or more mammary gland positive progenitor-cell markers compared to the expression level of said mammary gland positive progenitor-cell markers in EBs not treated with RA.

Where BMP4 and RA are added to the culture medium in the methods for producing a mammalian milk like product as described anywhere herein, the EBs generated in the methods as described anywhere herein express one or more mammary gland positive progenitor-cell markers. In some embodiments, said one or more mammary gland positive progenitor-cell markers are selected from EpCAM, CD49f, MUC1 and GATA3.

In some embodiments, the EBs have increased expression of one or more mammary gland positive progenitor-cell markers compared to the expression level of said mammary gland positive progenitor-cell markers in EBs not treated with BMP4.

In some embodiments, at least 10%, 20%, 30%, 40%, 50%, 60%, 70%, 80% or more of the EBs express one or more mammary gland positive progenitor-cell markers. In some embodiments, at least 10%, 20%, 30%, 40%, 50%, 60%, 70%, 80% or more of the EBs express two or more mammary gland positive progenitor-cell markers. In some embodiments, said mammary gland positive progenitor-cell markers are selected from EpCAM, CD49f, MUC1 and GATA3.

In some embodiments, at least 35% of EBs express EpCAM and CD49f mammary gland positive progenitor-cell markers. In some embodiments, at least 60% of EBs express EpCAM and CD49f mammary gland positive progenitor-cell markers at the middle differentiation stage. In some embodiments, at least 35% of EBs express EpCAM and CD49f mammary gland positive progenitor-cell markers at the pre-induction stage. In some embodiments, at least 40% of EBs express EpCAM and CD49f mammary gland positive progenitor-cell markers at the post-induction stage.

In some embodiments, at least 15% of EBs express MUC1 and EpCAM mammary gland positive progenitor-cell markers. In some embodiments, at least 20% of EBs express MUC1 and EpCAM mammary gland positive progenitor-cell markers at the middle differentiation stage. In some embodiments, at least 15% of EBs express MUC1 and EpCAM mammary gland positive progenitor-cell markers at the pre-induction stage. In some embodiments, at least 5% of EBs express MUC1 and EpCAM mammary gland positive progenitor-cell markers at the post-induction stage.

In some embodiments, at least 20% of EBs express GATA3 and EpCAM mammary gland positive progenitor-cell markers. In some embodiments, at least 50% of EBs express GATA3 and EpCAM mammary gland positive progenitor-cell markers at the middle differentiation stage. In some embodiments, at least 25% of EBs express GATA3 and EpCAM mammary gland positive progenitor-cell markers at the pre-induction stage. In some embodiments, at least 15% of EBs express GATA3 and EpCAM mammary gland positive progenitor-cell markers at the post-induction stage.

In some embodiments, the middle differentiation stage is day 25. The pre-induction stage is day 35 and the post-induction stage is day 42. In some embodiments, the middle differentiation stage is between day 20. The pre-induction stage is day 26 and the post-induction stage is day 31.

In some embodiments, the EBs express one or more milk-specific bioactive markers. In some embodiments, said milk-specific bioactive marker is osteopontin (OPN).

In some embodiments, the EBs have increased expression of one or more milk-specific bioactive markers compared to the expression level of said non-neuronal ectodermal markers in EBs not treated with BMP4 and RA.

Thus, as an example, provided herein is a method for producing a human milk like product, comprising generating lactocytes under step A) from human breast milk stem cells (hBSC), where such step A) comprises:
i) directing hBSCs to differentiate towards non-neural ectoderm cells by culturing them in an appropriate culture medium (for example MammoCult medium) and BMP4 as described anywhere herein, and after 10 days collecting mammospheres formed thereof; and
ii) growing such mammospheres in an appropriate system (for example a floating mixed gel culture system as described in Hassiotou F. et al. Stem Cells. 2012) for at least 10 days to generate lactocytes,
wherein the mammospheres (EBs) have increased expression of one or more mammary gland positive progenitor-cell markers compared to the expression level of said mammary gland positive progenitor-cell markers in mammospheres (EBs) not treated with BMP4.

Thus, as an example, provided herein is a method for producing a human milk like product, comprising generating lactocytes under step A) from human breast milk stem cells (hBSC), where such step A) comprises:
i) directing hBSCs to differentiate towards non-neural ectoderm cells by culturing them in an appropriate culture medium (for example MammoCult medium) and BMP4 as described anywhere herein, and after 6 days collecting mammospheres formed thereof; and
ii) growing such mammospheres in an appropriate system comprising RA (for example a floating mixed gel culture system as described in Hassiotou F. et al. Stem Cells. 2012) for less than 10 days to generate lactocytes,
wherein the mammospheres (EBs) have increased expression of one or more mammary gland positive progenitor-cell markers compared to the expression level of said mammary gland positive progenitor-cell markers in mammospheres (EBs) not treated with BMP4 and RA.

It will be understood that any method or method step disclosed herein may be conducted in 3D suspension culture rather than using a membrane matrix as a support. Thus, in some embodiments, during all the differentiation procedure, cells are maintained in suspension culture.

### Step B—Expressing a Human Breast Milk Like Product

In one embodiment of the present invention, the method comprises expressing the human milk like product from mammary like organoids derived from human adult breast milk stem cells (hBSCs), preferably prepared according to step A)

Expressing human milk like products preferably occurs upon induction of expression of the human milk like product from such lactocytes and/or mammary-like gland organoids.

In one embodiment, lactating lactocytes are induced by applying a specific medium (for example EpiCult B) supplemented with lactogenic factors (for example prolactin, hydrocortisone, and insulin).

Particularly, the human milk like product obtained from mammary like organoids derived from human breast milk stem cells (BSCs) preferably prepared according to step A), contains bioactives of human milk, selected from the group comprising or consisting of proteins, lipids or oligosaccharides, preferably human milk oligosaccharides, etc. Inventors particularly managed to identify inter alia oligosaccharides (lactose), lipids (C12:0, C16:0, C18:0, C18:1 n-9, C18:2 fatty acids) and proteins (lactoferrin, alphalactalbumin).

In one embodiment of the present invention, the human milk like product obtained from mammary like organoids derived from human breast milk stem cells (BSCs) is a standard human milk product. In another embodiment of the present invention, the human milk like product obtained from mammary like organoids derived from human breast milk stem cells (BSCs) is a non-standard human milk product.

### Step C—Further Treatments to Produce Modified Human Breast Milk Like Product

In one optional embodiment of the present invention, the method comprises an additional step C) which is performed on the human milk like product obtainable from step B) and which comprises performing an additional treatment on such product to provide a modified human milk like product.

In one embodiment, the additional treatment performed on the human breast milk like product may be selected in the group consisting of: a purification step, an isolation process, an extraction process, a fractionation step, an enrichment process, an enzymatic treatment, the addition of further components (for example which can't be expressed by the human mammary gland organoid (such as for example Immunoglobulins, probiotic, vitamins and/or minerals) or combinations thereof.

### Human Milk Like Products

### Standard Human Breast Milk Like Product

In one embodiment of the present invention, the human breast milk like product is a standard human breast milk like product, i.e., comprises the same components as human breast milk of a well-nourished mother.

The benefits of breast feeding are well known in the scientific literature and the possibility have access to a standard human breast milk like product allows its use for a number of equally well known health benefits.

In such embodiment, the standard human breast milk like product can be used as a substitute of breastfeeding under circumstances where real breastfeeding is not possible.

In such embodiment, the standard human breast milk like product is intended to be used for example to support longer breastfeeding experience for women who have less milk or who stop to produce milk after 6 months from birth.

Similarly, the standard human breast milk like product is intended to be used for example to allow breastfeeding even under circumstances where sicknesses compromise real breastfeeding from the mother.

In another embodiment, the standard human breast milk like product is intended to be used under circumstances whereby breastmilk production would not naturally be initiated, for example if an infant is adopted.

In one embodiment, the standard human milk like product according to the present invention is not the product of human breast milk lactation as occurring in nature.

In one embodiment, the standard human breast milk like product is for use in providing optimal nutrition for infant.

In one embodiment, the standard human breast milk like product is for use in providing healthy growth in infants.

In one embodiment, the standard human breast milk like product is for use in preventing infection, obesity and promoting immunity development in infants.

In one embodiment, the standard human breast milk like product is a non modified human breast milk like product.

In another embodiment, the standard human breast milk like product is a modified human breast milk like product.

In one embodiment, the standard human milk like product according to the present invention comprises: proteins, lipids, carbohydrates, vitamins and minerals.

In another embodiment, the standard human milk like product according to the present invention comprises: proteins, lipids, carbohydrates, vitamins, minerals and bioactives.

In one embodiment, the standard human milk like product according to the present invention comprises: proteins, lipids (including linoleic acid and alpha-linolenic acid), carbohydrates, Vitamins (including Vitamin A, Vitamin D3, Vitamin E, Vitamin K, Thiamin, Riboflavin, Niacin, Vitamin B6, Vitamin B12, Pantothenic acid, folic acid, myoinositol and L-carnitine.

In a further embodiment, the standard human milk like product according to the present invention also comprises at least one bioactive selected in the group consisting of: growth factors, cytokines, probiotics, extracellular vesicles (e.g. milk fat globules and or exosomes), bioactives from exosome (for example miRNA) and secretory IgA.

Such standard human breast milk like product may be prepared according to the method of the present invention for example by including a step C) of addition of growth factors, cytokines, probiotics, extracellular vesicles (e.g. milk fat globules and or exosomes), bioactives from exosomes (for example miRNA) and secretory IgA).

In one embodiment, the standard human breast milk like product contains probiotics. Such standard human breast milk like product may be prepared according to the method of the present invention for example by including a step C) of addition of probiotics (for example B. Lactis, B. Infantis, L. ramnhosus) which can be obtained from several commercially available sources.

In such embodiment, the standard human breast milk like product may be used for optimizing gastro intestinal function and/or promoting Immunity.

In one embodiment, the standard human breast milk like product contains secretory IgA and probiotics.

Such standard human breast milk like product may be prepared according to the method of the present invention for example by including a step C) of addition of a combination of probiotics and secretory IgA which may be prepared as described for example in patent applications WO2009/156301 and WO2009/156367 which are hereby incorporated by reference. In such embodiment, the standard human breast milk like product may be used for preventing Immunoglobulin deficiency and/or in the prevention of recurrent infection in infants and young children.

### Non-Standard Human Breast Milk Like Product

In one embodiment of the present invention, the human milk like product can have altered ratios and concentrations of components found naturally in human breast milk of a well-nourished mother. This is referred to herein as a “non-standard milk like product”.

In one embodiment, the non standard human milk like product according to the present invention may be selected in the group consisting of a milk fortifier, a supplement, and/or a human breast milk replacer adapted for special purposes.

Human Milk fortifiers and Human milk bioactive supplements

In one embodiment, the method of the present invention provides for a non-standard human breast milk like product which may be used to fortify human breast milk naturally obtained from a nursing mother or to fortify infant formulas.

In another embodiment, the method of the present invention provides for a non-standard human breast milk like product which may be used as a supplement for infants or young children in need thereof.

In such embodiments non standard human breast milk like product may be used for providing healthy growth and/or to reduce the risk of developing a disease typically associated to specific conditions in an infant or young child (such as for example asthma, allergy, cognitive alterations) and/or to promote catch up growth, development of immunity, protection from infections.

Remarkably, the human origin of the constituents (especially bioactive constituents) in such fortifiers or supplements combined with the fact that they are according to the method of the invention, is supposed to provide to such constituents, an intact or higher functionality.

The non-standard human breast milk like product intended to be used as a fortifier. Such non-standard human breast milk like product intended to be used as a fortifier may be prepared according to the method of the present invention for example by including a step C) of isolation and/or enrichment of certain bioactives from the non-modified human breast milk like product obtainable from step B). Such isolation step may be performed via classical fractionation, enrichment and/or purification of the non-modified human breast milk like product obtainable from step B).

The non-standard human breast milk like product intended to be used as a supplement may comprise one or more bioactives selected in the group consisting of: human milk oligosaccharides (for example 2FL, 3FL, LNT, LnNT, DiFI, 6SL and/or 3SL), lipids, growth factors (for example epidermal growth factor (EGF), heparin binding epidermal growth factor), cytokines (for example transforming growth factor-beta 2 (TGFbeta-2), IL-1. IL-2, IL-6, IL-10, IL-18, interferon gamma (INF-gamma), TNF-alpha), extracellular vesicles (e.g. milk fat globules and or exosomes), exosome comprising microRNAs and antimicrobial/protecting bioactives (for example lactoferrin, lysozyme, lactadherine). Such non-standard human breast milk like product intended to be used as a supplement may be prepared according to the method of the present invention for example by including a step C) of isolation of the bioactives from the non-modified human breast milk like product obtainable from step B). Such isolation step may be performed via classical fractionation, enrichment and/or purification of the non-modified human breast milk like product obtainable from step B).

In one embodiment, the non-standard human breast milk like product is a supplement or milk fortifier which contains fucosylated human milk oligosaccharides, for example 2FL and/or 3FL. Such supplement or milk fortifier is for use in completing the profile of human breast milk of women who do not secrete fucosylated oligosaccharides because of the inactivity of their FUT2 gene.

Such non-standard human breast milk like product intended to be used as a fortifier or supplement may be prepared according to the method of the present invention for example by including a step C) of isolation and/or enrichment of fucosylated oligosaccharides (for example 2FL and or 3FL) from the non-modified human breast milk like product obtainable from step B).

In such embodiment, the standard human breast milk like product may be used for optimizing gastro intestinal function and/or promoting Immunity.

Human Breast Milk Like Product for Infants with Genetic Diseases

In one embodiment, the non standard human breast milk like product according to the present invention may be adapted to address the specific need of infants who are born with a genetic disease.

### Galactossemia

In such embodiment, the non standard human breast milk like product may be adapted to the needs on infants suffering from Galactossemia. Galactossemia is a rare genetic disease that affects babies' ability to metabolize galactose.

In such embodiment, the non standard human breast milk like product should be deprived of lactose and/or lactose containing saccharides. In such embodiment non standard human breast milk like product may be used for providing healthy growth to the infants affected by galactossemia.

In one embodiment, a non standard human breast milk like product deprived of lactose and/or lactose containing saccharides may be obtained according to the method of the present invention by including a step C) of enzymatic treatment (lactase treatment), or of membrane fractionation and ultrafiltration of the non-modified human breast milk like product obtainable from step B).

In another embodiment, a non standard human breast milk like product deprived of lactose and/or lactose containing saccharides may be obtained according to the method of the present invention by using under step A) alpha-lactalbumin deficient hBSCs.

### Phenyl Keturonia

In such embodiment, the non standard human breast milk like product may be adapted to the needs on infants suffering from Phenyl Keturonia (PKU). PKU is due to absent or dysfunctional phenylalanine hydroxylase, which converts phenylalanine to tyrosine. Untreated, it leads to severe mental retardation due to brain toxicity.

In such embodiment, the non standard human breast milk like product should be deprived or depleted of phenylalanine. In such embodiment non standard human breast milk like product may be used for providing healthy growth to the infants affected by PKU. In one embodiment, the non standard human breast milk like product is depleted of phenyl alanine in such a way that phenylalanine content is kept below 20 mg/kg body weight of the subject receiving it.

In one embodiment, a non standard human breast milk like product depleted or deprived of phenylalanine may be obtained according to the method of the present invention by including a step C) of enzymatic treatment (protein hydrolysis) or of filtration of the non-modified human breast milk like product obtainable from step B).

In one embodiment, a non standard human breast milk like product depleted of phenylalanine may be obtained according to the method of the present invention by including a step C) of enzymatic treatment (protein hydrolysis) or of filtration of the non-modified human breast milk like product obtainable from step B).

In another embodiment, a non standard human breast milk like product depleted of phenylalanine may be obtained according to the method of the present invention by providing in step B) a culture medium providing limited or zero amounts of phenylalanine, such as for example a culture medium containing Glycomacropeptide (GMP) from whey.

It should be appreciated that the various aspects and embodiments of the detailed description as disclosed herein are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

As used in this detailed description and the appended claims, the singular forms “a,” “an” and “the” include plural referents unless the context clearly dictates otherwise.

### Additional Embodiments of the Invention

In a first additional embodiment, there is provided:

S1. A method for producing a mammalian milk like product comprising:
A) Generating lactocytes mammary-like gland organoids derived from mammalian adult breast milk stem cells (mBSCs);
B) Secreting the mammalian milk like product from such lactocytes from mammalian adult breast milk stem cells (mBSCs).

S2. A method according to statement 1 for producing a non standard human milk like product comprising:
A) Generating lactocytes mammary-like gland organoids derived from human adult breast milk stem cells (hBSCs);
B) Secreting the human milk like product from such lactocytes from human adult breast milk stem cells (hBSCs),
   wherein such non standard human milk like product comprises one or more of the nutrients or bioactives selected from the group consisting of proteins, peptides, lipids, carbohydrates, Vitamins, minerals, choline, myoinositol, L-carnitine, growth factors, cytokines, probiotics, extracellular vesicles, bioactives from exosome and secretory IgA.

S3. A method according to statement 1 or 2 which optionally comprises a step C) whereby the human milk like product of statement 1 is further treated to obtain a modified human milk like product.

S4. A method according to statement 3 wherein step C) is selected in the group consisting of: a purification step, an isolation process, an extraction process, a fractionation step, an enrichment process, an enzymatic treatment, the addition of further components and combinations thereof.

S5. A method for producing a human milk like product according to anyone of statements 2 to 4 wherein culture conditions according to step A) are adapted to generate lactocytes derived from human adult breast milk stem cells (hBSCs) capable to secret a non-standard human milk like product.

S6. A method according to anyone of statements 3 to 5 wherein a modified human milk like product is obtained under step C) by adding to the human milk like product of step B) one or more human breast milk components that are not secreted by the lactocytes in step B).

S7. A method according to anyone of statements 1 to 6 wherein the lactocytes are part of a mammary gland-like organoid structure generated under step A).

S8. A method according to anyone of statements 1 to 7 wherein the human milk like product consist or comprises bioactives selected form the group consisting of: oligosaccharides, lipids and proteins.

S9. A method according to anyone of statements 1 to 8 wherein the human milk like product consist or comprises bioactives selected form the group consisting of: lactose, C12:0, C16:0, C18:0, C18:1 n-9, C18:2, lactoferrin and alphalactalbumin.

S10. A human milk like product which is obtainable according to the method described in anyone of statements 2 to 9.

S11. A human milk like product according to statement 10 which comprises or consists of bioactives selected form the group consisting of: oligosaccharides, lipids and proteins.

S12. A human milk like product according to statement 11 which comprises or consist of bioactives selected from the group consisting of: lactose, C12:0 fatty acid, C16:0 fatty acid, C18:0 fatty acid, C18:1 n-9 fatty acid, C18:2 fatty acid, lactoferrin and alphalactalbumin.

S13. A human milk like product according to anyone of statements 10 to 12 for use in therapy.

S14. Use of a human breast milk like product according to anyone of statements 10 to 12 as a breast feeding substitute.

In a second additional embodiment, there is provided:

S1. A method of producing a population of mammary gland cells, comprising:
i) culturing human breast milk stem cells (hBSCs) in a culture medium comprising bone morphogenic protein 4 (BMP4) to generate embryoid bodies (EBs), and
ii) growing the EBs to generate a population of mammary cells.

S2. The method of statement 1, wherein the culturing step i) comprises culturing the hBSCs in MammoCult medium and BMP4, in a 3D-suspension culture system, for example 3D-suspension condition, thereby directing the hBSCs to differentiate towards non-neural ectoderm cells, optionally for at least 12 days.

S3. The method of statements 1 or 2, wherein the growing step ii) comprises growing the formed EBs in a 3D embedding system, for example a mixed floating gel composed of matrix protein such as Matrigel and/or Collagen I for at least 30 days, for example for 32 days, to generate lactocytes.

S4. The method of any one of statements 1 to 3, wherein the mammary gland cells are human mammary gland cells.

S5. The method of any one of statements 1 to 4, wherein BMP4 is added to the culture medium between day 0 and day 10, preferably between day 0 and day 3, where day 0 is the time point where the hBSCs are first added to the culture medium.

S6. The method of any one of statements 1 to 5, wherein BMP4 is added to the culture medium for 3 days.

S7. The method of any one of statements 1 to 6, wherein BMP4 is added to the culture medium in a concentration of 5 to 20 ng/ml, preferably 5 ng/ml, 10 ng/ml or 20 ng/mL.

S8. The method of any one of statements 1 to 7, wherein the EBs express one or more mammary gland positive progenitor-cell markers, optionally selected from EpCAM, CD49f, MUC1 and GATA3.

S9. The method of any one of statements 1 to 8, wherein the EBs have increased expression of one or more mammary gland positive progenitor-cell markers compared to the expression level of said mammary gland positive progenitor-cell markers in EBs not treated with BMP4.

S10. The method of any one of statements 1 to 9, wherein at least 10%, 20%, 30%, 40%, 50%, 60%, 70%, 80% or more of the EBs express one or more mammary gland positive progenitor-cell markers, optionally wherein at least 50% of EBs express EpCAM and CD49f mammary gland positive progenitor-cell markers, optionally wherein at least 15% of EBs express MUC1 and CD49f mammary gland positive progenitor-cell markers, optionally wherein at least 20% of EBs express MUC1 and EpCAM mammary gland positive progenitor-cell markers, and/or optionally wherein at least 15% of EBs express GATA3 mammary gland positive progenitor-cell markers.

S11. The method of any one of statements 1 to 10, wherein the EBs express one or more non-neuronal ectodermal markers, optionally selected from TFAP2A and TFAP2C.

S12. The method of any one of statements 1 to 11, wherein the EBs have increased expression of one or more non-neuronal ectodermal markers of at least 2-fold compared to the expression level of said non-neuronal ectodermal markers in EBs not treated with BMP4, optionally at least a 3 to 15-fold increase.

S13. The method of any one of statements 1 to 12, wherein the EBs have decreased expression of one or more neuronal ectodermal markers of at least 0.5-fold compared to the expression level of said neuronal ectodermal markers in EBs not treated with BMP4, optionally wherein said neuronal ectodermal markers are selected from PAX6, OXT2 and SOX11.

S14. The method of any one of statements 1 to 13, wherein the EBs express one or more milk-specific bioactive markers, optionally osteopontin (OPN).

S15. The method of any one of statements 1 to 14, wherein the EBs have increased expression of OPN of at least 2-fold compared to the expression level of OPN in EBs not treated with BMP4, optionally at least a 4 to 18-fold increase.

S16. The method of any one of statements 1 to 15, wherein the mammary gland cells form lactocyte mammary-like gland organoids.

S17. The method of any one of statements 1 to 16, wherein the mammary gland cells generate increased expression of milk-specific bioactive markers compared to mammary gland cells that are not treated with BMP4, optionally wherein the mammary gland cells generate at least a 2-fold increase in expression of milk-specific bioactive markers compared to mammary gland cells that are not treated with BMP4, optionally wherein said markers are selected from estrogen related receptor alpha (ESRRA), Keratin 14 (KRT14) and MUC1.

S18. Use of BMP4 for increasing the differentiation efficiency of human breast milk stem cells (hBSCs) into mammary-gland progenitor cells in a differentiation protocol.

S19. A method for producing a mammalian milk like product, comprising:
C) Generating lactocyte mammary-like gland organoids derived from human breast milk stem cells (hBSC);
D) Secreting the mammalian milk like product from said lactocytes,
wherein step A) comprises culturing the hBSCs in a culture medium comprising BMP4.

S20. The method of statement 19, wherein the method is for producing a human milk like product,
wherein step A) further comprises:
i) directing hBSCs to differentiate towards non-neural ectoderm cells by culturing them in an appropriate culture medium comprising BMP4, for example MammoCult medium and BMP4, in an appropriate 3D culture system, for example 3D-suspension condition, for at least 12 days and
ii) growing the formed mEBs (mammospheres) in an appropriate 3D embedding system, for example a mixed floating gel composed of matrix protein such as Matrigel and/or Collagen I for at least 30 days, for example for 32 days, to generate lactocytes.

S21. The method according to statement 20 wherein step A)i) is defined as follows:
i) generation of embryoid bodies (EBs) from hBSCs by incubation in standard hBSC medium E8 comprising DMEM/F12, L-ascorbic acid-2-phosphate magnesium, sodium selenium, FGF2, insulin, NaHCO₃ and transferrin, TGFβ1 or NODAL or in medium mTeSR™ for two days, and producing mEBs (mammospheres) highly enriched in non-neural ectodermal cells by incubation of EBs in complete MammoCult medium comprising the basal medium, proliferation supplement and supplemented with BMP4, heparin, and hydrocortisone for 10 days, and wherein
step A)ii) is distinguished into further substeps and comprises the following steps: ii), iii) and iv):
ii) incubation of mEBs (mammospheres) in complete EpiCultB medium supplemented with EpiCult proliferation supplement and Parathyroid hormone (pTHrP) for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days, and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days.

S22. The method according to statement 20 wherein step A)i) is defined as follows:
i) generation of embryoid bodies (EBs) from hBSCs by incubation in standard hBSC medium E8 comprising DMEM/F12, L-ascorbic acid-2-phosphate magnesium, sodium selenium, FGF2, insulin, NaHCO₃ and transferrin, TGFβ1 or NODAL for two days, and producing mEBs (mammospheres) highly enriched in non-neural ectodermal cells by incubation of EBs in MammoCultB medium supplemented with MammoCult proliferation supplement, hydrocortisone, heparin and BMP4 for 10 days, and wherein
step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement and Parathyroid hormone (pTHrP) for 5 days,
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days, and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days.

S23. The method of any one of statements 19 to 22, wherein Step A) comprises a method of any one of statements 1 to 17.

S24. A human milk like product which is obtainable according to the method described in anyone of statements 19 to 23.

S25. A human milk like product according to statement 24 for use in therapy.

S26. Use of a human milk like product according to statement 24 as a human milk substitute, optionally a breast-feeding substitute.

In a third additional embodiment, there is provided:

S1. A method of producing a population of mammary gland cells, comprising:
i) culturing human breast milk stem cells (hBSCs) in a culture medium retinoic acid (RA) to generate embryoid bodies (EBs), and
ii) growing the EBs to generate a population of mammary cells.

S2. The method of statement 1, wherein the culturing step i) comprises culturing the hBSCs in MammoCult medium, in a 3D-suspension culture system, for example 3D-suspension condition, thereby directing the hBSCs to differentiate towards non-neural ectoderm cells, optionally for at least 12 days.

S3. The method of statements 1 or 2, wherein the growing step ii) comprises growing the EBs in a 3D embedding system comprising RA, for example a mixed floating gel composed of matrix protein such as Matrigel and/or Collagen I for at least 30 days, for example for 32 days.

S4. The method of statement 3, wherein step ii) is distinguished into further substeps and comprises the following steps: ii) and iii):
ii) incubation of mEBs (mammospheres) in complete EpiCultB medium supplemented with EpiCult proliferation supplement, Parathyroid hormone (pTHrP), and RA for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days.

S5. The method of statement 3, wherein step ii) is distinguished into further substeps and comprises the following steps: ii) and iii):
ii) embedding the formed mEBs (mammospheres) in a mixture of Matrigel and Collagen I floated in EpiCultB medium supplemented with EpiCult proliferation supplement, Parathyroid hormone (pTHrP), and RA for 5 days,
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days.

S6. The method of any one of statements 1 to 5, wherein the mammary gland cells are human mammary gland cells.

S7. The method of any one of statements 1 to 6, wherein RA is added to the culture medium between day 10 and day 15, where day 0 is the time point where the hBSCs are first added to the culture medium.

S8. The method of any one of statements 1 to 7, wherein RA is added to the culture medium for 5 days.

S9. The method of any one of statements 1 to 8, wherein RA is added to the culture medium in a concentration of 1 μM.

S10. The method of any one of statements 1 to 9, wherein the EBs express one or more mammary gland positive progenitor-cell markers, optionally selected from EpCAM, CD49f and MUC1.

S11. The method of any one of statements 1 to 10, wherein the EBs have increased expression of one or more mammary gland positive progenitor-cell markers compared to the expression level of said mammary gland positive progenitor-cell markers in EBs not treated with RA.

S12. The method of any one of statements 1 to 11, at least 10%, 20%, 30%, 40%, 50% or more of the EBs express one or more mammary gland positive progenitor-cell markers, optionally wherein at least 40% of EBs express EpCAM and CD49f mammary gland positive progenitor-cell markers, optionally wherein at least 10% of EBs express MUC1 and CD49f mammary gland positive progenitor-cell markers, and/or optionally wherein at least 15% of EBs express MUC1 and EpCAM mammary gland positive progenitor-cell markers.

S13. The method of any one of statements 1 to 12, wherein the EBs have increased cell viability compared to EBs not treated with RA.

S14. The method of any one of statements 1 to 13, wherein the EBs have at least 85% or more viability, preferably 95% or more.

S15. The method any one of statements 1 to 14, wherein the mammary gland cells form lactocyte mammary-like gland organoids.

S16. Use of RA for increasing the differentiation efficiency of human breast milk stem cells (hBSCs) into mammary-gland progenitor cells in a differentiation protocol.

S17. A method for producing a mammalian milk like product, comprising:
A) Generating lactocyte mammary-like gland organoids derived from human breast milk stem cells (hBSC);
B) Secreting the mammalian milk like product from said lactocytes,
wherein step A) comprises culturing the hBSCs in a culture medium comprising RA.

S18. The method of statement 16, wherein the method is for producing a human milk like product,
wherein step A) further comprises:
i) directing hBSCs to differentiate towards non-neural ectoderm cells by culturing them in an appropriate culture medium, for example MammoCult medium, in an appropriate 3D culture system, for example 3D-suspension condition, for at least 12 days and
ii) growing the formed mEBs (mammospheres) in an appropriate 3D embedding system comprising RA, for example a mixed floating gel composed of matrix protein such as Matrigel and/or Collagen I and RA for at least 30 days, for example for 32 days, to generate lactocytes.

S19. The method according to statement 18 wherein step A)i) is defined as follows:
i) generation of embryoid bodies (EBs) from hBSCs by incubation in standard hBSC medium E8 comprising DMEM/F12, L-ascorbic acid-2-phosphate magnesium, sodium selenium, FGF2, insulin, NaHCO₃ and transferrin, TGFβ1 or NODAL or in medium mTeSR™ for two days, and producing mEBs (mammospheres) highly enriched in non-neural ectodermal cells by incubation of EBs in complete MammoCult medium comprising the basal medium, proliferation supplement and supplemented with heparin, and hydrocortisone for 10 days, and wherein
step A)ii) is distinguished into further substeps and comprises the following steps: ii), iii) and iv):
ii) incubation of mEBs (mammospheres) in complete EpiCultB medium supplemented with EpiCult proliferation supplement, Parathyroid hormone (pTHrP) and RA for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days, and iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days.

S20. The method according to statement 18 wherein step A)i) is defined as follows:
i) generation of embryoid bodies (EBs) from hBSCs by incubation in standard hBSC medium E8 comprising DMEM/F12, L-ascorbic acid-2-phosphate magnesium, sodium selenium, FGF2, insulin, NaHCO₃ and transferrin, TGFβ1 or NODAL for two days, and producing mEBs (mammospheres) highly enriched in non-neural ectodermal cells by incubation of EBs in MammoCultB medium supplemented with MammoCult proliferation supplement, hydrocortisone and heparin for 10 days, and wherein step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement, Parathyroid hormone (pTHrP) and RA for 5 days,
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days, and
iv) induction of milk protein expression by incubating mEBs (mammospheres) in proliferation supplement, EpiCultB medium supplemented with EpiCult hydrocortisone, insulin, FBS, prolactin, progesterone and β-estradiol for 7 days.

S21. The method of any one of statements 17 to 20, wherein Step A) comprises a method of any one of statements 1 to 15.

S22. A human milk like product which is obtainable according to the method described in anyone of statements 17 to 20.

S23. A human milk like product according to statement 22 for use in therapy.

S24. Use of a human milk like product according to statement 22 as a human milk substitute, optionally a breast-feeding substitute.

In a fourth additional embodiment, there is provided:

S1. A method of producing a population of mammary gland cells, comprising:
i) culturing mammalian breast milk stem cells (mBSCs) in a culture medium comprising bone morphogenic protein 4 (BMP4) and/or retinoic acid (RA) to generate embryoid bodies (EBs), and
ii) growing the EBs to generate a population of mammary cells.

S2. The method of statement 1, wherein the culture medium comprises bone morphogenic protein 4 (BMP4) and retinoic acid (RA). S3. The method of statement 1 or 2, wherein the culturing step i) comprises culturing the mBSCs in MammoCult medium and BMP4, in a 3D-suspension culture system, for example 3D-suspension condition, thereby directing the hBSCs to differentiate towards non-neural ectoderm cells, optionally for at least 12 days.

S4. The method of statement 2, wherein the culturing step i) comprises culturing the hBSCs in MammoCult medium and BMP4, in a 3D-suspension culture system, for example 3D-suspension condition, thereby directing the mBSCs to differentiate towards non-neural ectoderm cells, optionally for no more than 8 days.

S5. The method of any one of statements 1 to 3, wherein the growing step ii) comprises growing the formed EBs in a 3D embedding system comprising RA, for example a mixed floating gel composed of matrix protein such as Matrigel and/or Collagen I for at least 30 days, for example for 32 days, to generate lactocytes.

S6. The method of statements 2 or 4, wherein the growing step ii) comprises growing the formed EBs in a 3D embedding system comprising RA, for example a mixed floating gel composed of matrix protein such as Matrigel and/or Collagen I for no more than 25 days, to generate lactocytes.

S7. The method of statement 5, wherein step ii) is distinguished into further substeps and comprises the following steps: ii) and iii):
ii) incubation of mEBs (mammospheres) in complete EpiCultB medium supplemented with EpiCult proliferation supplement, Parathyroid hormone (pTHrP), and RA for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days.

S8. The method of statement 6, wherein step ii) is distinguished into further substeps and comprises the following steps: ii) and iii):
ii) embedding the formed mEBs (mammospheres) in a mixture of Matrigel and Collagen I floated in EpiCultB medium supplemented with EpiCult proliferation supplement, Parathyroid hormone (pTHrP), and RA for 5 days,
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 15 days.

S9. The method of any one of statements 1 to 8, wherein the mammary gland cells are human mammary gland cells.

S10. The method of any one of statements 1 to 9, wherein BMP4 is added to the culture medium between day 0 and day 10, preferably between day 0 and day 3, where day 0 is the time point where the hBSCs are first added to the culture medium.

S11. The method of any one of statements 1 to 10, wherein BMP4 is added to the culture medium for 3 days.

S12. The method of any one of statements 1 to 11, wherein RA is added to the culture medium between day 10 and day 15, optionally between day 6 and day 11, where day 0 is the time point where the hBSCs are first added to the culture medium.

S13. The method of any one of statements 1 to 12, wherein RA is added to the culture medium for 5 days.

S14. The method of any one of statements 1 to 13, wherein BMP4 is added to the culture medium in a concentration of 5 to 20 ng/ml, preferably 5 ng/ml, 10 ng/ml or 20 ng/ml.

S15. The method of any one of statements 1 to 14, wherein RA is added to the culture medium in a concentration of 1 μM.

S16. The method of any one of statements 1 to 15, wherein the EBs express one or more mammary gland positive progenitor-cell markers, optionally selected from EpCAM, CD49f, MUC1 and GATA3.

S17. The method of any one of statements 2 to 15, wherein the EBs have increased expression of one or more mammary gland positive progenitor-cell markers compared to the expression level of said mammary gland positive progenitor-cell markers in EBs not treated with BMP4 and RA.

S18. The method of any one of statements 1 to 16, wherein at least 10%, 20%, 30%, 40%, 50%, 60%, 70%, 80% or more of the EBs express one or more mammary gland positive progenitor-cell markers, optionally wherein at least 35% of EBs express EpCAM and CD49f mammary gland positive progenitor-cell markers, optionally wherein at least 15% of EBs express MUC1 and EpCAM mammary gland positive progenitor-cell markers, and/or optionally wherein at least 20% of EBs express EpCAM and GATA3 mammary gland positive progenitor-cell markers.

S19. The method of any one of statements 1 to 18, wherein the EBs express one or more milk-specific bioactive markers, optionally osteopontin (OPN).

S20. The method of any one of statements 1 to 19, wherein the EBs have increased expression one or more milk-specific bioactive markers, optionally increased expression of osteopontin (OPN).

S21. The method of any one of statements 1 to 20, wherein the EBs have increased secretion one or more milk-specific bioactive markers, optionally increased secretion of osteopontin (OPN).

S22. The method of any one of statements 1 to 21, wherein the mammary gland cells form lactocyte mammary-like gland organoids.

S23. Use of BMP4 and/or RA for increasing the differentiation efficiency of mammalian breast milk stem cell (mBSCs) into mammary-gland progenitor cells in a differentiation protocol.

S24. A method for producing a mammalian milk like product, comprising:
A) Generating lactocyte mammary-like gland organoids derived from mammalian breast milk stem cells (mBSC);
B) Secreting the mammalian milk like product from said lactocytes,
wherein step A) comprises culturing the hBSCs in a culture medium comprising BMP4 and/or RA.

S25. The method of statement 24, wherein the culture medium comprises bone morphogenic protein 4 (BMP4) and retinoic acid (RA).

S26. The method of statement 24 or 25, wherein step A) is between a 30-day and 45-day process, optionally less than a 45-day process, optionally less than a 44-day process, optionally less than a 35-day process, optionally less than a 31-day process.

S27. The method of any one of statements 24 to 26, wherein the method is for producing a human milk like product, wherein step A) further comprises:
i) directing mBSCs to differentiate towards non-neural ectoderm cells by culturing them in an appropriate culture medium comprising BMP4, for example MammoCult medium and BMP4, in an appropriate 3D culture system, for example 3D-suspension condition, for at least 12 days and
ii) growing the formed mEBs (mammospheres) in an appropriate 3D embedding system comprising RA, for example a mixed floating gel composed of matrix protein such as Matrigel and/or Collagen I and RA for at least 30 days, for example for 32 days, to generate lactocytes.

S28. The method of statement 25 or 26, wherein the method is for producing a human milk substitute product,
wherein step A) further comprises:
i) directing mBSCs to differentiate towards non-neural ectoderm cells by culturing them in an appropriate culture medium comprising BMP4, for example MammoCult medium and BMP4, in an appropriate 3D culture system, for example 3D-suspension condition, for no more than 8 days and
ii) growing the formed mEBs (mammospheres) in an appropriate 3D embedding system comprising RA, for example a mixed floating gel composed of matrix protein such as Matrigel and/or Collagen I and RA for no more than 25 days, to generate lactocytes.

S29. The method according to statement 27 wherein step A)i) is defined as follows:
i) generation of embryoid bodies (EBs) from hBSCs by incubation in standard mBSC medium E8 comprising DMEM/F12, L-ascorbic acid-2-phosphate magnesium, sodium selenium, FGF2, insulin, NaHCO₃ and transferrin, TGFβ1 or NODAL or in medium mTeSR™ for two days, and producing mEBs (mammospheres) highly enriched in non-neural ectodermal cells by incubation of EBs in complete MammoCult medium comprising the basal medium, proliferation supplement and supplemented with BMP4, heparin, and hydrocortisone for 10 days, and wherein
step A)ii) is distinguished into further substeps and comprises the following steps: ii), iii) and iv):
ii) incubation of mEBs (mammospheres) in complete EpiCultB medium supplemented with EpiCult proliferation supplement and Parathyroid hormone (pTHrP) and RA for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days, and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days.

S30. The method according to statement 28 wherein step A)i) is defined as follows:
i) generation of embryoid bodies (EBs) from hBSCs by incubation in standard mBSC medium E8 comprising DMEM/F12, L-ascorbic acid-2-phosphate magnesium, sodium selenium, FGF2, insulin, NaHCO₃ and transferrin, TGFβ1 or NODAL or in medium mTeSR™ for two days, and producing mEBs (mammospheres) highly enriched in non-neural ectodermal cells by incubation of EBs in complete MammoCult medium comprising the basal medium, proliferation supplement and supplemented with BMP4, heparin, and hydrocortisone for 6 days, and wherein
step A)ii) is distinguished into further substeps and comprises the following steps: ii), iii) and iv):
ii) incubation of mEBs (mammospheres) in complete EpiCultB medium supplemented with EpiCult proliferation supplement and Parathyroid hormone (pTHrP) and RA for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 15 days, and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 5 days.

S31. The method according to statement 27 wherein step A)i) is defined as follows:
i) generation of embryoid bodies (EBs) from hBSCs by incubation in standard mBSC medium E8 comprising DMEM/F12, L-ascorbic acid-2-phosphate magnesium, sodium selenium, FGF2, insulin, NaHCO₃ and transferrin, TGFβ1 or NODAL for two days, and producing mEBs (mammospheres) highly enriched in non-neural ectodermal cells by incubation of EBs in MammoCultB medium supplemented with MammoCult proliferation supplement, hydrocortisone, heparin and BMP4 for 10 days, and wherein
step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement and Parathyroid hormone (pTHrP) and RA for 5 days,
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 20 days, and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days.

S32. The method according to statement 28 wherein step A)i) is defined as follows:
i) generation of embryoid bodies (EBs) from hBSCs by incubation in standard mBSC medium E8 comprising DMEM/F12, L-ascorbic acid-2-phosphate magnesium, sodium selenium, FGF2, insulin, NaHCO₃ and transferrin, TGFβ1 or NODAL for two days, and producing mEBs (mammospheres) highly enriched in non-neural ectodermal cells by incubation of EBs in MammoCultB medium supplemented with MammoCult proliferation supplement, hydrocortisone, heparin and BMP4 for 6 days, and wherein step A)ii) is distinguished into further substeps and comprises the following steps ii), iii) and iv):
ii) embedding the formed mEBs (mammospheres) in a mixture of Matrigel and Collagen I floated in EpiCultB medium supplemented with EpiCult proliferation supplement and Parathyroid hormone (pTHrP) and RA for 5 days,
iii) promotion of branch and alveolar differentiation and mammary cell specification by incubating embedded mEBs (mammospheres) in EpiCultB medium supplemented with EpiCult proliferation supplement, hydrocortisone, insulin, FGF10 and HGF for 15 days, and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 5 days.

S33. The method of any one of statements 24 to 32, wherein Step A) comprises a method of any one of statements 1 to 22.

S34. The method of statement 24-26 and 33, wherein Step A) is conducted in 3D suspension culture conditions.

S35. A human milk like product which is obtainable according to the method described in anyone of statements 24 to 34.

S36. A human milk like product according to statement 35 for use in therapy.

S37. Use of a human milk like product according to statement 35 as a human milk substitute, optionally a breast-feeding substitute.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

### EXPERIMENTAL SECTION

### Example 1

Cultivation and Differentiation of hBSCs into Lactocytes

The objective of the present inventors was to isolate hBSC from human breast milk cells collected from donors, and collecting supernatants for analysis during their differentiation under mammary differentiation conditions (conditions described in Hassiotou F. et al. Stem Cells. 2012) to demonstrate the lactocyte functionality on the bases of the nutrients thereby secreted.

### Materials

### Breastmilk Donation Samples

Breastmilk donations, obtained after obtaining signed Informed Consent forms were used up to 3 hours from expression.

### Formulation of Spheroid Formation Medium

500 ml MammoCult medium kit was supplemented with 3% Pen-Strep and 2 μL/ml fungizone (Table 1).

TABLE 1
Spheroid formation medium

| Component | Manufacturer |
|---|---|
| MammoCult medium kit, 500 ml | Stem Cell |
| | Technologies |
| Amphotericin B solution (fungizone) | Sigma |
| Pen-Strep (antibiotic-antimycotic solution) | Biological |
| | industries |

### Formulation of Mammary Differentiation Medium

500 ml RPMI 1640 with L-glutamine was supplemented with 20% FBS, 4 μg/ml insulin, 20 ng/ml EGF, 0.5 μg/ml hydrocortisone, 5% Pen-Strep, and 2 μL/ml fungizone (Table 2)

TABLE 2
Mammary differentiation medium

| Component | Manufacturer |
|---|---|
| RPMI 1640 with L- | Biological industries |
| glutamine, 500 ml | |
| Fetal bovine serum | Biological industries |
| (FBS) | |
| Insulin | Gibco |
| Epidermal growth | Peprotech |
| factor (EGF) | |
| hydrocortisone | Sigma |
| Amphotericin B | Sigma |
| solution (fungizone) | |
| Pen-Strep (antibiotic- | Biological industries |
| antimycotic solution) | |

### Experimental Design

1.1 Isolation of Human Breast Milk Cells (hBMC)
Each breastmilk donation was diluted with PBS (phosphate buffered saline) (1:1) and centrifuge (800×g for 20 minutes at 20° C.).
Fat layer and liquid part skim milk were removed, and cell pellet was washed three times in PBS (300×g for 5 minutes at 20° C.).
Cell viability was determined by flow cytometry.
   1.2 Staining of Human Breast Milk Cells (hBMC) with Embryonic Stem Cell Markers
A sample of 100 μL hBMC from each donation was stained for viability and with antibodies against embryonic stem cell markers (according to each antibody manufacturer's instructions):

TABLE 3
Flow cytometry antibodies

| Target | Manufacturer |
|---|---|
| OCT4- AlexaFluor488 | BioLegend |
| SOX2 - PE | BioLegend |
| NANOG- AlexaFluor647 | BioLegend |
| SSEA4- PE/Cy7 | BioLegend |
| TRA-1-60- | BioLegend |
| PerCP/Cyanine5.5 | |
| Viability 405/452 Fixable | Miltenyi Biotec |
| Dye | |

Cells were analyzed by FACS (LSRII-new cell analyzer with five lasers, Becton Dickinson)
   1.3 Culture hBMC as Spheroids
The remaining hBMC from each donation were cultured in ultra-low binding 6-well plates (Corning) in spheroid formation medium (3 ml/well) (see Table 1) at 37° C. and 5% CO2.
   1.4 Culture of hBMC Under Mammary Differentiation Conditions (3-4 Weeks) and Supernatant Collection
Primary hBMC spheroids was incubated in mammary differentiation medium in 37° C. and 5% CO2 for 4 weeks in tissue culture 24-well plates (Corning), at 1-3 wells per each breast milk donation (1 ml medium per well) (see Table 2). Spheroids were collected and centrifuge (300 g. 5 minutes). Medium was discarded and 1 ml trypsin was added for 5 minutes. Thereafter the cells were centrifuges, trypsin was removed and 1 ml of mammary medium was added to the cells.
Every week, the supernatant was collected from each well and stored at (−20±5° C.).
Fresh mammary differentiation medium was added to each well.

### 1.5 Alpha-Lactalbumin and Lactoferrin Detection by ELISA

Supernatants were analyzed for Alpha-lactalbumin (Cloud clone, Cat #SEB018Hu) and Lactoferrin (Cloud clone, Cat #SEA780Hu) by ELISA according to each ELISA kit manufacturer's instructions.

### Results

### Flow Cytometry:

As reported in Table 4, hBMC from all three donors expressed all five markers. TRA-1-160 expression was the highest, 23.5%-33.4% from all viable cells. SSEA4, SOX2, and NANOG had a wider expression range, 2.5%-21%, 6.2%-22.1%, and 0.9%-22.2% from all viable cells, respectively. OCT4 expression was the lowest, 0.1%-2.7% from all viable cells.

TABLE 4
Flow cytometry results

| | % from viable cells | | |
|---|---|---|---|
| Donor 1 | | | Stained- |
| Target | Unstained | Stained | Unstained |
| Viability | | 63% | |
| TRA-1- | 0.5 | 26.7 | 26.2 |
| 160 | | | |
| SSEA4 | 0.1 | 20.8 | 20.7 |
| OCT4 | 0.5 | 3.2 | 2.7 |
| SOX2 | 0.2 | 19.7 | 19.5 |
| NANOG | 0.2 | 10.4 | 10.2 |
| | % from viable cells | | |
| Donor 2 | | | Stained- |
| Target | Unstained | Stained | Unstained |
| Viability | | 50% | |
| TRA-1- | 0.2 | 33.6 | 33.4 |
| 160 | | | |
| SSEA4 | 0.9 | 8.9 | 8 |
| OCT4 | 0.4 | 2.2 | 1.8 |
| SOX2 | 0.6 | 6.8 | 6.2 |
| NANOG | 0 | 0.9 | 0.9 |
| | % from viable cells | | |
| Donor 3 | | | Stained- |
| Target | Unstained | Stained | Unstained |
| Viability | | 77% | |
| TRA-1- | 0 | 23.5 | 23.5 |
| 160 | | | |
| SSEA4 | 0 | 2.5 | 2.5 |
| OCT4 | 0.1 | 0.2 | 0.1 |
| SOX2 | 0 | 22.1 | 22.1 |
| NANOG | 0.1 | 22.3 | 22.2 |

### 1.6 Culture Appearance:

After one week in mammocult medium in low-binding plates, hBMC formed spheroids. Once the cells are plated in culture plates in mammary medium, some of them attach to the surface and exhibit mammary-like morphology.

### 1.7 ELISA:

ELISA results of Lactoferrin and alpha-lactalbumin secretion from hBMC cultures in mammary medium after 1 week culture are reported in Table 7 here below for each donor.

TABLE 7

| Donor | 1 | 2 | 3 |
|---|---|---|---|
| Lactoferrin | 48 | 95 | 80 |
| (μg/mL) | | | |
| Lactalbumin | 0.8 | 5.5 | 4.8 |
| (μg/mL) | | | |

### Summary and Conclusions

hBMC expressed various levels of embryonic stem cell markers, confirming the presence of embryonic stem cells able to differentiate to various cell lineages.
Under mammary differentiation conditions, some of these embryonic stem cells acquired mammary like morphology, which was maintained for about two weeks.

Thus, under the conditions of the present study, mammary-like cells exhibited differentiation and were able to be cultured.

### Example 2

### Analysis of Lipids Secreted by Lactocytes

Method: The supernatants obtained at weeks 1 and 2 under procedure 1.4 as above described is analysed to investigate the presence of fatty acids contained in several lipid classes secreted by the lactocytes. A 7890A gas-chromatograph with a 7693 autosampler with preparative station module equipped with a fused-silica CP-Sil 88 capillary column (100% cyanopropylpolysiloxane; 100 m, 0.25 mm id, 0.25 mm film thickness is used with a split injector (1:25 ratio) heated at 250° C. and a flame-ionization detector operated at 300° C.

Preparation of FAMEs (fatty acids methyl esters) is performed by direct transesterification of sample with methanolic chloridric acid. Separation of FAMEs is performed using capillary gas chromatography-FID (GC).

Identification of FAMEs is done by retention time (RT) and comparison with an external standard. Quantification of FA is done by calculation using methyl C11:0 as internal standard. Transesterification performance of the method is controlled with TAG C13:0 as second internal standard.
Into a 10 mL screw cap glass test tube add 250 ml of supernatant sample.
Add 300 ml of internal standard FAME C11:0 solution
Add 300 ml of internal standard TAG C13:0 solution
Add 2 mL of methanol.
Add 2 mL of Methanol/HCl (3N).
Add 1 mL of hexane.
Firmly cap the test tubes and shake vigorously.
Heat at 100° C./60 min, shake occasionally.
Let it cool down to room temperature (about 15 min).
Add 2 ml of water.
Centrifuge at 1200 g for 5 min.
Filter the hexane phase (upper phase) trough Pasteur pipette containing glass wool and anhydrous sodium sulphate into a clean and empty GC vial.

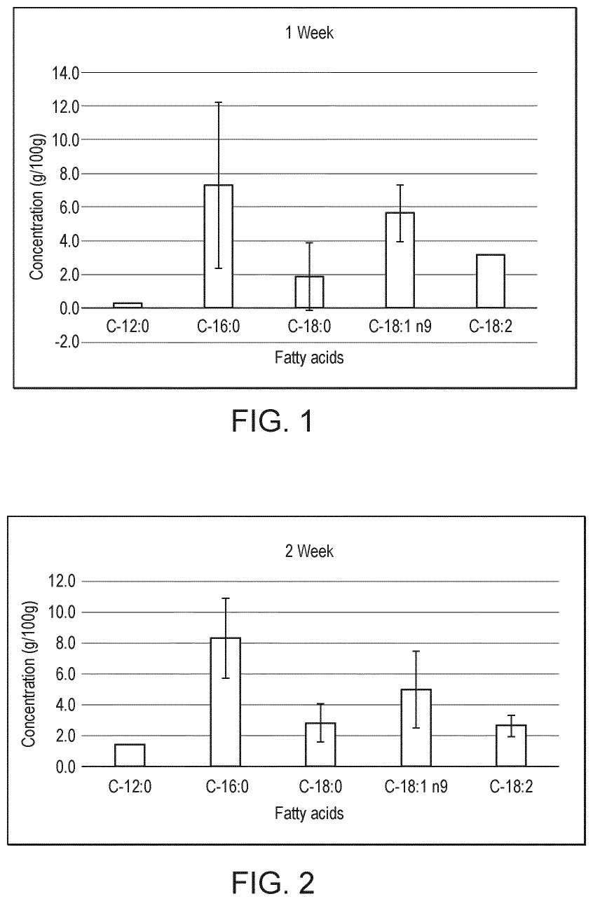

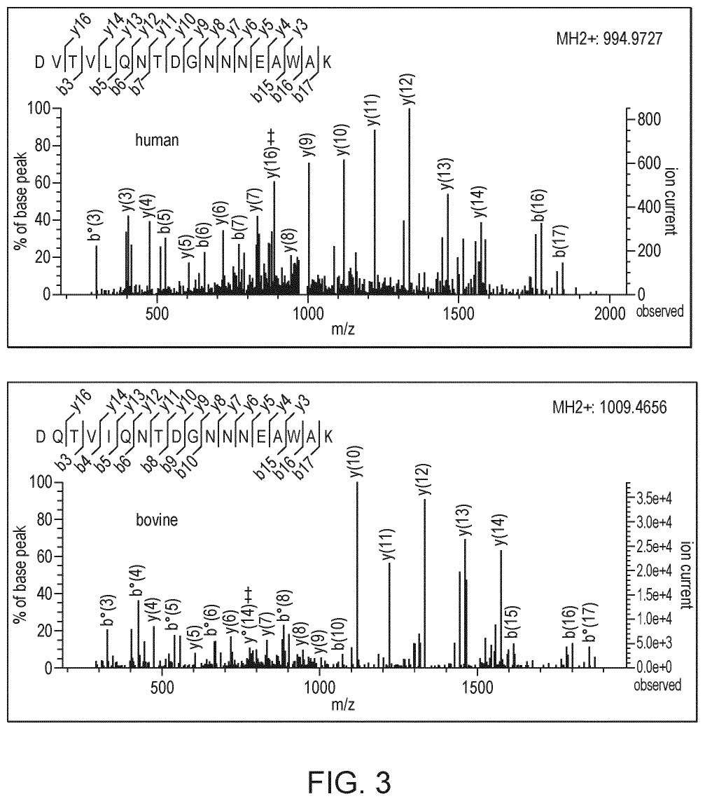

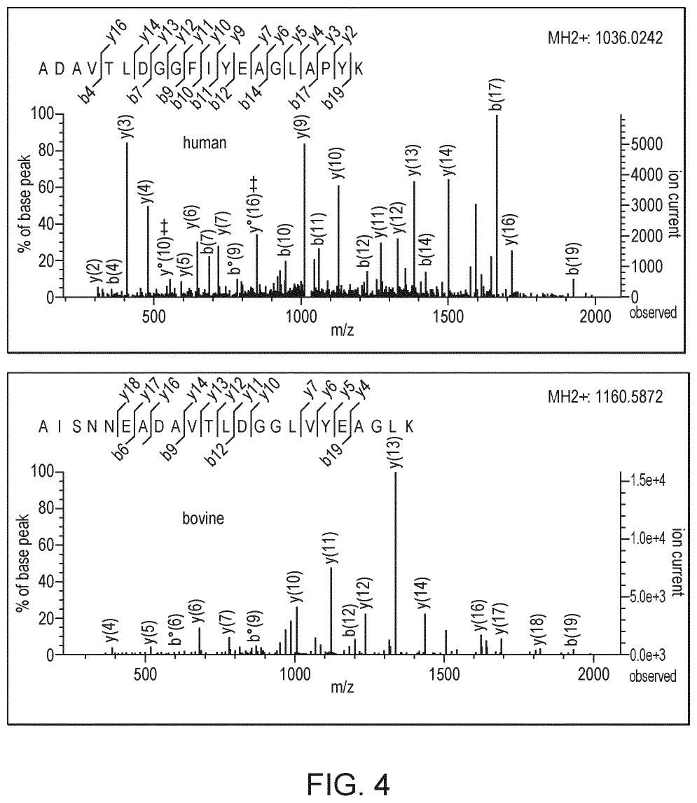

Results: Analysis revealed the presence of fatty acids as reported in table 5, FIG. 1 and Table 6, FIG. 2 for weeks 1 and 2 respectively; showing the functionality of lactocytes for lipids' production.

TABLE 5

| | Average all donors (week | |
|---|---|---|
| Fatty Acids | 1) | SD |
| C-12:0 | 0.3 | |
| C-16:0 | 7.3 | 4.9 |
| C-18:0 | 1.9 | 2.0 |
| C-18:1 n9 | 5.7 | 1.7 |
| C-18:2 | 3.2 | 0.6 |

TABLE 6

| | Average all donors | |
|---|---|---|
| Fatty acid | (week 2) | SD |
| C-12:0 | 1.4 | |
| C-16:0 | 8.3 | 2.6 |
| C-18:0 | 2.8 | 1.3 |
| C-18:1 n9 | 5.0 | 2.5 |
| C-18:2 | 2.6 | 0.7 |

### Example 3

### Analysis of Proteins in the Supernatant

Method: Identification of proteins in a supernatant sample obtained at week 1 as described above in 1.4 has been performed according to the procedure described by Dzieciatkowska et al. (Dzieciatkowska M., Hill R, and Hansen K C, Methods Mol Biol. 2014; 1156:53-66; GeLC-MS/MS Analysis of Complex Protein Mixtures). In brief, proteins in the culture supernatant were separated by one-dimensional SDS-PAGE analysis. After protein staining, detected protein bands were excised and in-gel digested, followed by separation and detection of the released peptides by LC-MS/MS analysis. The MS spectra were further processed by protein database searching using Mascot (Matrix Science). Stringent filter criteria were applied (peptide mass tolerance: 5 ppm; semi-tryptic cleavage; false discovery rate (FDR): 1%; keratin contamination filtered out) to detected human proteins in a high background of bovine proteins in the supernatant (due to the presence of fetal bovine serum in the culture medium).

Result: three distinct human milk proteins have been identified in this analysis, namely alpha-2-macroglobulin, inter-alpha-trypsin inhibitor heavy chain H2 and lactoferrin. For example, human lactoferrin was identified with 14 matching peptides which are uniquely different from corresponding homologous bovine lactoferrin peptides. Two examples of tandem MS peptide spectra matches of human and bovine peptide from lactoferrin are given below in FIGS. 3 & 4. These results confirm the presence of human milk proteins, e.g. lactoferrin, in the supernatant of the hBMC cultures.

### Example 4

### Analysis of Carbohydrates in the Supernatant

Method: supernatant sample obtained at week 1, 2 and 3 as described above in 1.4 was analysed for presence of lactose or human milk oligosaccharides following the procedure described by Austin and Benet for human milk [Austin, S.; Benet, T. Quantitative determination of non-lactose milk oligosaccharides. Analytica Chimica Acta 2018, 1010, 86-96]. Briefly, an aliquot of primary cell supernatant was mixed with a solution of laminaritriose before labelling with 2-aminobenzamide. After labelling, the samples were analysed with UHPLC and detected lactose or oligosaccharides quantified against a calibration curve of maltotriose assuming equimolar response factors.

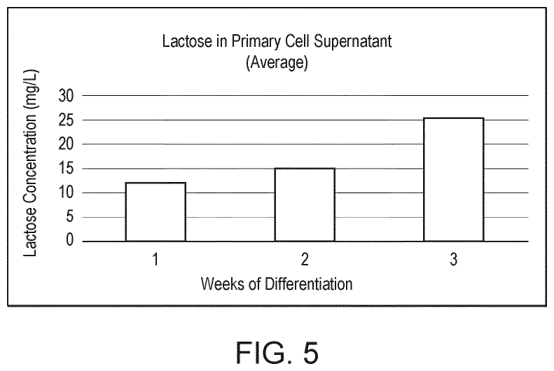

Results are reported in FIG. 5.

Results: Lactose was found in the primary cell supernatants at 1, 2 and 3 weeks of differentiation with concentrations varying between 10 and 25 mg/L.

### Example 5

Effect of Bone Morphogenetic Protein 4 (BMP4) on Non-Neural Ectoderm Lineage Differentiation of Induced Pluripotent Stem Cells (iPSCs) Using the 3D-Organoid Setting

Methods: The human-induced pluripotent stem cell (hiPSC) line 603 was purchased from Fujifilm Cellular Dynamics, Inc (FCDI) and used for the 3D-differentiaion of mammary gland progenitors. Briefly, 5.5 million dissociated iPSCs were plated in the 6 well-plates (ultra-low attachment) containing 4.5 mL aggregation media using a planar shaker platform (set at 95 RPM). BMP4 (314-BP-050, Bio-Techne AG) at different concentrations was added to the culture media between day 0 and day 3.

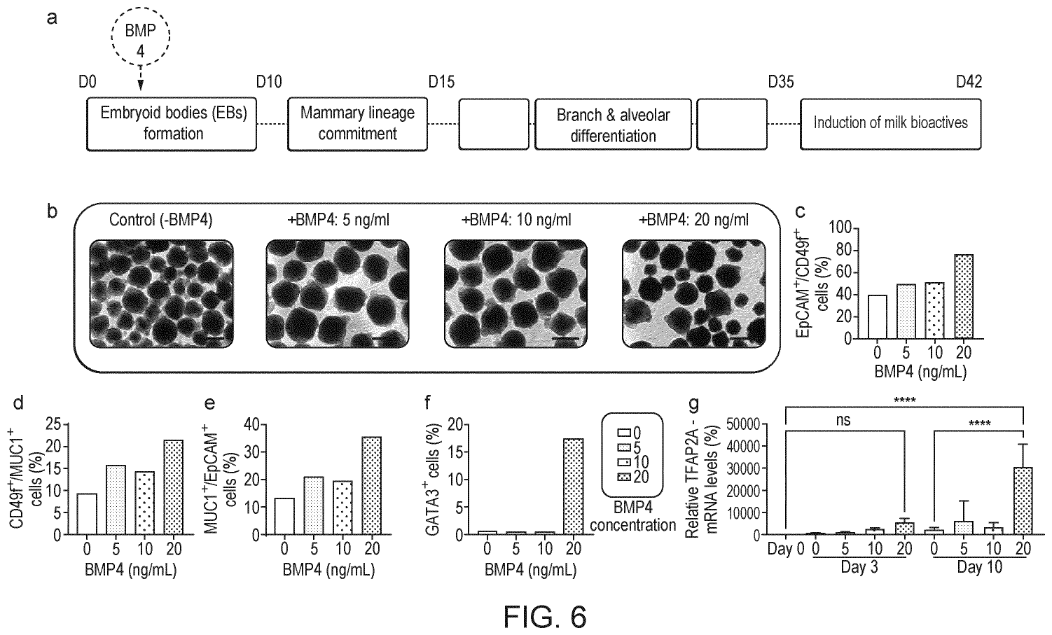

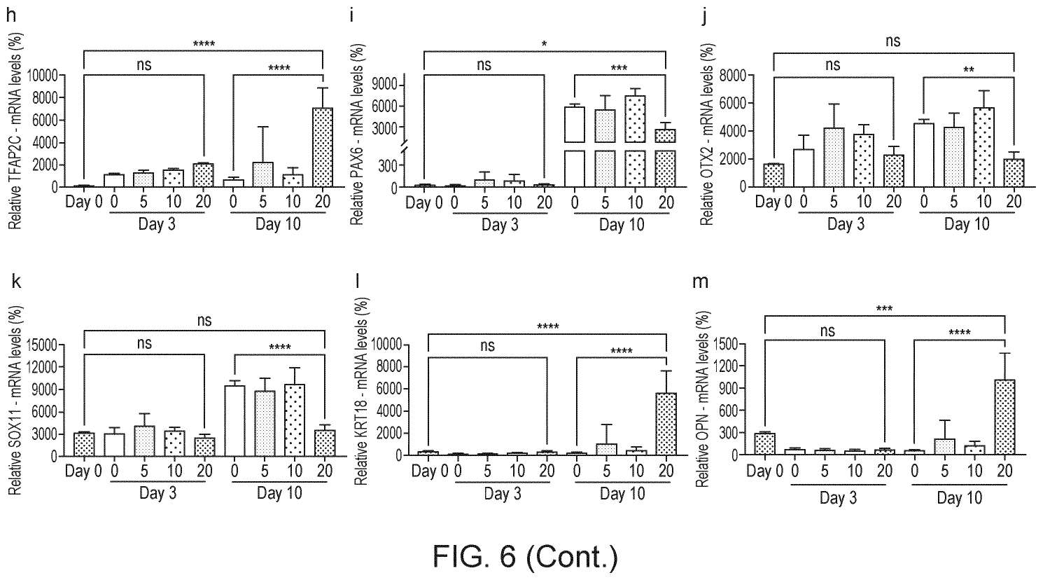

Results: iPSC cells were tested with different concentrations of BMP4 to induce non-neural ectodermal differentiation (FIG. 6a, b). 20 ng/ml of BMP4 significantly induced the expression of mammary gland progenitor markers such as EpCAM (CD326), CD49f, MUC1 (CD227) and GATA3 using flow cytometry quantification (FIG. 6c-f). Expression of human TFAP2A and TFAP2C (AP2 Gamma) as predominant non-neural ectodermal markers were increased at day 10 of differentiation using Nanostring analysis compared to the control sample (FIG. 6g-h). Moreover, expression levels of human specific neural ectoderm markers, such as paired box gene 6 (PAX6), orthodenticle homeobox 2 (OTX2) and SRY-box transcription factor 11 (SOX11) were decreased during differentiation (FIG. 6i-k). Lastly, BMP4 can induce the expression of the human luminal specific markers cytokeratin 18 (KRT18) and secreted phosphorylated milk glycoprotein osteopontin (OPN) (FIG. 6l-m).

In more detail, FIG. 6 shows the effect of bone morphogenetic protein 4 (BMP4) on non-neural ectoderm lineage differentiation of induced pluripotent stem cells (iPSCs) using the 3D-organoid setting. a, Scheme summarizing the procedure for the generation of mammary gland progenitors and BMP4 treatment between day 0 and day 3. b, Microscopic analysis of morphological changes after exposure to different concentration of BMP4. c-f, Flow cytometry quantification of the number of mammary gland positive progenitor-cells using different identified markers: EpCAM, CD49f, MUC1 and GATA3. g-h, RNA expression level of human TFAP2A and TFAP2C as predominant non-neural ectodermal markers. i-k, human PAX6, OTX2 and SOX11 as neural ectoderm markers. l, human KRT18 as a luminal marker. m, human OPN as a secreted phosphorylated milk glycoprotein.

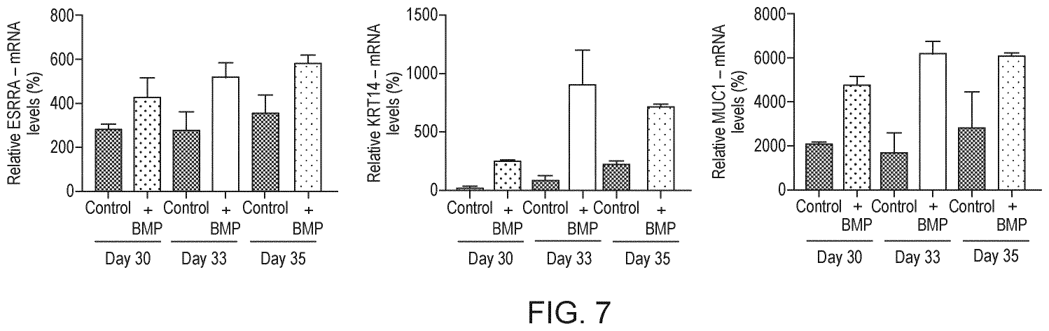

In more detail, FIG. 7 shows effect of bone morphogenetic protein 4 (BMP4) on estrogen related receptor alpha (ESRRA), Keratin 14 (KRT14) and MUC1 RNA expression level between day 30 and 35 of mammary-gland 3D-differentiation.

These results demonstrate that BMP4 steers stem cell commitment towards the non-neural ectodermal lineage and surprisingly significantly increases mammary gland progenitor cell specification.

### Example 6

The effect of BMP4 at the end-stage of 3D-mammary gland differentiation of iPSC cells was assessed using estrogen related receptor alpha (ESRRA), Keratin 14 (KRT14) and MUC1 for their expression profiles. Surprisingly, BMP4 increased the RNA expression levels of all the genes at day 30, 33 and 35 compared to the control sample (FIG. 7).

These results demonstrate that BMP4 significantly increases milk bioactive expression in mammary gland cells derived from the differentiation of stem cells.

### Example 7

Effect of Retinoic Acid (RA), Bovine Serum Albumin (BSA) and Fetal Bovine Serum (FBS) on Mammary Gland Specific Lineage Differentiation of Induced Pluripotent Stem Cells (iPSCs) Using the 3D-Organoid Setting

Methods: The human-induced pluripotent stem cell (hiPSC) line 603 was purchased from Fujifilm Cellular Dynamics, Inc (FCDI) and used for the 3D-differentiation of mammary gland progenitors. Briefly, 5.5 million dissociated iPSCs were plated in the 6 well-plates (ultra-low attachment) containing 4.5 mL aggregation media using a planar shaker platform (set at 95 RPM). RA (1 μM, 10552611, Fisher Scientific), BSA (5%, A7030-50G, Sigma-Aldrich) and FBS (5%, 10270106, Gibco) between day 10 and day 15.

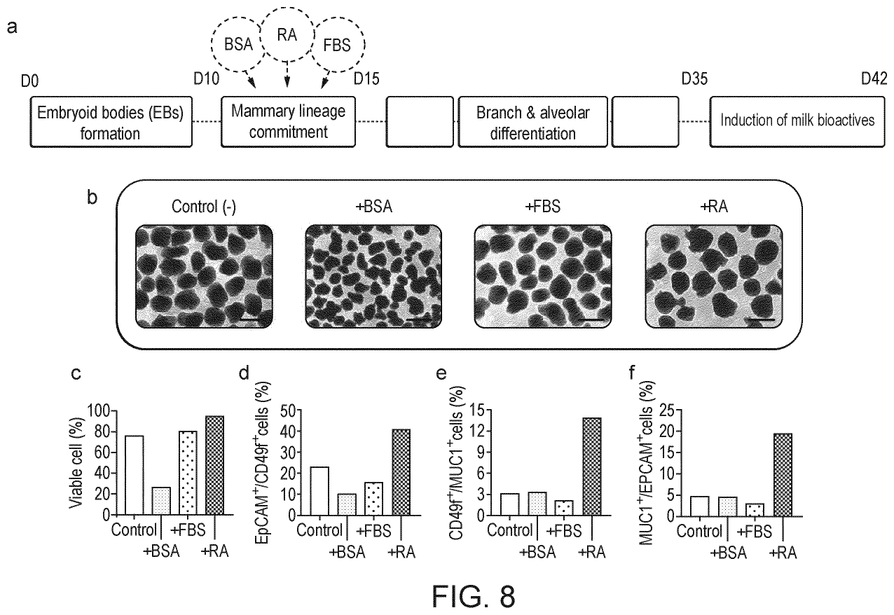

Results: The effect of retinoic acid (RA), bovine serum albumin (BSA) and fetal bovine serum (FBS) was tested in the differentiation of iPSCs towards the mammary epithelial lineage, following non-neural ectoderm specification. RA-induced organoids show a transition in morphology from spheroid-shaped towards budding organoids which potentially emulate alveologenesis in human mammary glands (FIG. 8b). Progenitor cells viability is significantly higher in the RA condition compared to BSA and FBS (FIG. 8c). Flow cytometry quantification of the number of mammary gland progenitor cells following RA treatment revealed a significant increase on EPCAM (CD326), CD49f and MUC1 (CD227) expression levels (FIG. 8d-f).

In more detail, FIG. 8 shows the effect of retinoic acid (RA), bovine serum albumin (BSA) and fetal bovine serum (FBS) on mammary gland specific lineage differentiation of induced pluripotent stem cells (iPSCs) using the 3D-organoid setting. a, Scheme summarizing the procedure for the generation of mammary gland progenitors and the application of RA, BSA and FBS between day 10 and day 15. b, Microscopic analysis of morphological changes after exposure to RA, BSA and FBS. c, Calcein-AM and ethidium homodimer-1 based flow cytometric assessment of cell viability across different conditions. d-f, Flow cytometry quantification of the number of mammary gland progenitor cells using epithelial markers: EpCAM, CD49f and MUC1.

Consequently, RA can enhance epithelial progenitor cell differentiation in the early stage of mammary organoid development.

### Example 8

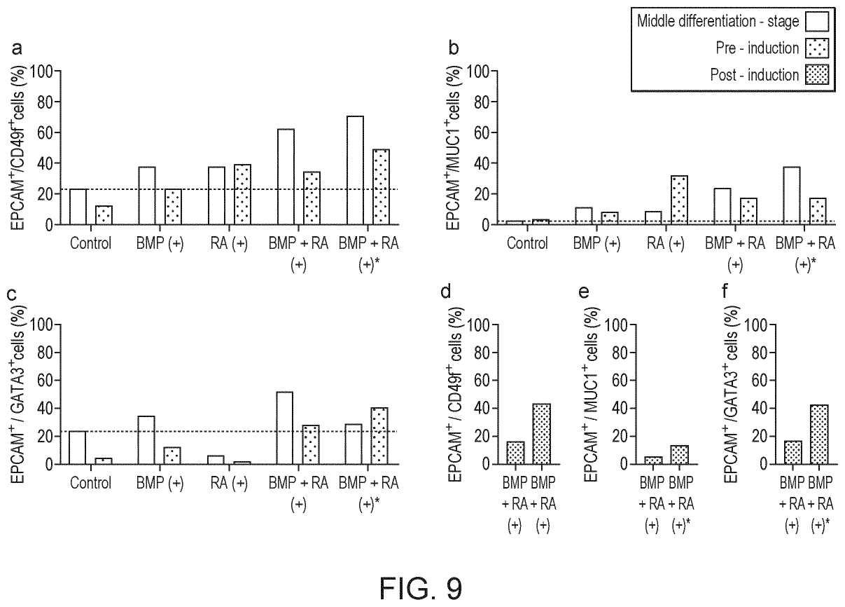

In order to generate milk bioactives that closely resemble those found in human breast milk, different methods using bone morphogenetic protein 4 (BMP4) and retinoic acid (RA) in 42 days and 31 days as a shortened time-course protocol were established in a 3D-platform using iPSCs derived mammary gland organoids as a biomimetic model of the human mammary gland (FIG. 9).

3D-iPSC cells in 42 days protocol and shortened time-course protocol (31 days*—* describes shortened-modified protocols (31 days) including BMP4 and RA combinations) using 20 ng/ml of BMP4 and 1 μM of RA in single or combination format compared to the control condition can induce the expression of mammary gland progenitor markers such as EpCAM (CD326), CD49f, MUC1 (CD227) and GATA3 using flow cytometry quantification or maintain their expression profile during the differentiation time-course and/or pre-induction stages (FIG. 9a-c) and post-induction period (FIG. 9d-f).

In more detail, FIG. 9 shows combinatorial effects of bone morphogenetic protein 4 (BMP4) and retinoic acid (RA) on mammary-gland differentiation of induced pluripotent stem cells (iPSCs) using a 3D-organoid model. a-c, Flow cytometry quantification of the number of mammary gland positive progenitor-cells using several identified markers: EpCAM, CD49f, MUC1 and GATA3 during the differentiation time-course (day 25 (for control protocols) and day 20 (for shortened time-course protocol)) and pre-induction stages (day 35 (for control protocols including BMP4 and RA), and day 26 (for shortened time-course protocol)) and d-f, post-induction period (days 42 (for control protocols including BMP4 and RA) and 31 (for shortened time-course protocol)). * describes shortened-modified protocols (31 days) including BMP4 and RA combinations.

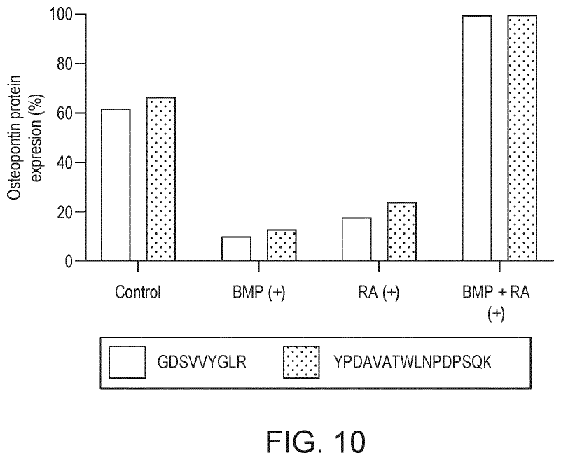

Human osteopontin peptides—GDSVVYGLR and -YPDAVATWLNPDPSQK were specifically detected in the cell culture supernatant by LC-MS/MS (FIG. 10).

In more detail, FIG. 10 shows Liquid chromatography-mass spectrometry (LC-MS/MS) based targeted proteomic analysis of osteopontin protein secretion. Osteopontin protein secretion was detected in the media of cultures of iPS-derived mammary-gland cells using control differentiation method, or with bone morphogenetic protein 4 (BMP4), retinoic acid (RA) and in the combination of the two factors.

These results demonstrate that BMP4 and RA in combination significantly increase mammary gland progenitor marker and milk bioactive expression and secretion in mammary gland cells derived from the differentiation of stem cells.
It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A method of producing a population of mammary gland cells, comprising:
i) culturing mammalian breast milk stem cells (mBSCs) in a culture medium comprising bone morphogenic protein 4 (BMP4) and/or retinoic acid (RA) to generate embryoid bodies (EBs), and
ii) growing the EBs to generate a population of mammary cells.

2. The method of claim 1, wherein the culture medium comprises bone morphogenic protein 4 (BMP4) and retinoic acid (RA).

3. The method of claim 1, wherein the culturing step i) comprises culturing the mBSCs in MammoCult medium and BMP4, in a 3D-suspension culture system, for example 3D-suspension condition, thereby directing the hBSCs to differentiate towards non-neural ectoderm cells.

4. (canceled)

5. The method of claim 1, wherein the growing step ii) comprises growing the formed EBs in a 3D embedding system comprising RA, for example a mixed floating gel composed of matrix protein such as Matrigel and/or Collagen I for at least 30 days to generate lactocytes.

6-8. (canceled)

9. The method of claim 1, wherein the mammary gland cells are human mammary gland cells.

10. The method of claim 1, wherein BMP4 is added to the culture medium between day 0 and day 10, where day 0 is the time point where the hBSCs are first added to the culture medium.

11. (canceled)

12. The method of claim 1, wherein RA is added to the culture medium between day 10 and day 15, where day 0 is the time point where the hBSCs are first added to the culture medium.

13. (canceled)

14. The method of claim 1, wherein BMP4 is added to the culture medium in a concentration of 5 to 20 ng/mL.

15. (canceled)

16. The method of claim 1, wherein the EBs express one or more mammary gland positive progenitor-cell markers.

17-18. (canceled)

19. The method of claim 1, wherein the EBs express one or more milk-specific bioactive markers.

20. The method of claim 1, wherein the EBs have increased expression one or more milk-specific bioactive markers.

21. The method of claim 1, wherein the EBs have increased secretion one or more milk-specific bioactive markers.

22. The method of claim 1, wherein the mammary gland cells form lactocyte mammary-like gland organoids.

23. (canceled)

24. A method for producing a mammalian milk like product, comprising:
C) Generating lactocyte mammary-like gland organoids derived from mammalian breast milk stem cells (mBSC);
D) Secreting the mammalian milk like product from said lactocytes,
wherein step A) comprises culturing the hBSCs in a culture medium comprising BMP4 and/or RA.

25. The method of claim 24, wherein the culture medium comprises bone morphogenic protein 4 (BMP4) and retinoic acid (RA).

26. The method of claim 24, wherein step A) is between a 30-day and 45-day process.

27. The method of claim 24, wherein the method is for producing a human milk like product,
wherein step A) further comprises:
i) directing mBSCs to differentiate towards non-neural ectoderm cells by culturing them in an appropriate culture medium comprising BMP4, for example MammoCult medium and BMP4, in an appropriate 3D culture system, for example 3D-suspension condition, for at least 12 days and
ii) growing the formed mEBs (mammospheres) in an appropriate 3D embedding system comprising RA, for example a mixed floating gel composed of matrix protein such as Matrigel and/or Collagen I and RA for at least 30 days, for example for 32 days, to generate lactocytes.

28. (canceled)

29. The method according to claim 1 wherein step A)i) is defined as follows:
i) generation of embryoid bodies (EBs) from hBSCs by incubation in standard mBSC medium E8 comprising DMEM/F12, L-ascorbic acid-2-phosphate magnesium, sodium selenium, FGF2, insulin, NaHCO₃ and transferrin, TGFβ1 or NODAL or in medium mTeSR™ for two days, and producing mEBs (mammospheres) highly enriched in non-neural ectodermal cells by incubation of EBs in complete MammoCult medium comprising the basal medium, proliferation supplement and supplemented with BMP4, heparin, and hydrocortisone for 10 days, and wherein
step A)ii) is distinguished into further substeps and comprises the following steps: ii), iii) and iv):
ii) incubation of mEBs (mammospheres) in complete EpiCultB medium supplemented with EpiCult proliferation supplement and Parathyroid hormone (pTHrP) and RA for 5 days,
iii) promotion of branch and alveolar differentiation and mammary cell specification by incubating mEBs (mammospheres) in EpiCultB medium supplemented with EpiCult proliferation supplement, hydrocortisone, insulin, FGF10 and HGF for 20 days, and
iv) induction of milk protein expression by incubating mEBs (mammospheres) in EpiCultB medium supplemented with EpiCult proliferation supplement, hydrocortisone, insulin, FBS, prolactin, progesterone and β-estradiol for 7 days.

30-37. (canceled)


SEQUENCE LISTING (2 sequences)

SEQ ID NO: 1 - protein, 9 aa, Homo sapiens
GDSVVYGLR

SEQ ID NO: 2 - protein, 16 aa, Homo sapiens
YPDAVATWLNPDPSQK